(19) United States
(12) Patent Application Publication
(10) Pub. No.: US 2025/0205673 A1
(43) Pub. Date: Jun. 26, 2025

(54) NITRIC OXIDE GENERATION SYSTEMS AND METHODS

(71) Applicant: Nanjing Novlead Biotechnology Co., Ltd., Nanjing (CN)

(72) Inventors: Wen Mao, Nanjing (CN); Yuyan Zhang, Nanjing (CN); Qing Wu, Nanjing (CN); Yuheng Zhang, Nanjing (CN); Qiyu Sun, Nanjing (CN); Liangyuan Ji, Nanjing (CN)

(21) Appl. No.: 18/993,946

(22) PCT Filed: Jul. 14, 2022
(86) PCT No.: PCT/CN2022/105787

§ 371 Date: Jan. 13, 2025

(51) Int. Cl. B01J 19/24; B01J 4/00; B01J 14/00; B01J 19/00; C01B 21/24

(52) U.S. Cl. CPC B01J 19/24; B01J 4/004; B01J 14/00; B01J 19/0006; C01B 21/24; B01J 2204/002; B01J 2219/00164; B01J 2219/00186

(57) ABSTRACT

Embodiments of nitric oxide (NO) generation apparatuses, systems, and methods are provided. In some embodiments, an NO generation system may include a first reservoir configured to contain a first solution, a second reservoir configured to contain a second solution, a first flow generator configured to be in fluid communication with the first reservoir, a second flow generator that may be configured to be in fluid communication with the second reservoir, and a housing configured to contain a reaction chamber and a gas chamber. The first solution may include a nitrite source. The second solution may include an acidic solution. The reaction chamber may be in fluid communication with at least one of the first flow generator and the second flow generator. The NO generation system may include a carrier gas source disposed upstream of and in fluid communication with the gas chamber.

### TECHNICAL FIELD

The present disclosure relates to nitric oxide generation field, and more particularly to nitric oxide generation systems and methods.

### BACKGROUND

Nitric oxide (NO) is a gaseous signaling molecule that plays important roles in many physiological and pathological processes. NO gas may diffuse through cell membranes without an intermediary transport mechanism and thus can signal neighboring cells or tissue in an efficient and speedy manner. For example, NO gas produced by vascular endothelial cells can signal the surrounding vascular smooth muscles to relax, resulting in vasodilation and increased blood flow. NO gas may also participate in electron transfer and redox reactions in cellular biochemical events in human bodies. NO gas may elicit various physiological effects, such as endothelium-dependent vasodilation, by activating guanylyl cyclase. Endogenous NO gas may be produced in response to host viral and microbial infections and may suppress replication of pathogens.

Administering exogenously produced NO gas to a wound site of a body may promote wound healing by improving the body's response to damage, pain, and infection. For example, introducing NO gas into to a wound site may improve local blood microcirculation and inhibit viral and bacterial infection at the wound site. For example, exogenously produced NO gas may be used to treat foot or limb wounds in diabetic patients, such as diabetic foot ulcer (DFU). DFU is characterized by an inability to self-repair in a timely and orderly manner. DFU often progress to infections, osteomyelitis, and gangrene, subsequently resulting in toe amputations, leg amputations, and death. DFU represents a significant public health problem throughout the world. Conventional treatments of DFU include various types of wound dressings, antibiotics, wound healing growth factors, autografts and allografts, use of wheelchairs and crutches to remove mechanical pressure, and amputation. However, these treatments are often expensive, ineffective within a reasonable time period, or dramatically reduce life quality. Low cost, portable, and non-invasive treatments of DFU are needed to reduce morbidity and mortality of DFU patients and reduce burden to the healthcare system.

Controlled administration of exogenously produced NO gas may provide a viable treatment of DFU. However, in clinical settings, high-pressure gas tanks or cylinders are used for providing NO gas. Such tanks are of significant size and weight and are typically secured to a wheeled delivery device or cart, typically to be placed at the bedside in a crowded intensive care unit. Using such heavy and bulky gas tanks may pose safety risks to the patients and healthcare workers. For example, patients and healthcare workers may be exposed to toxic nitrogen dioxide (NO₂) formed during system setup or due to potential NO gas leaks from damaged regulators, valves, or supply lines. Healthcare workers may also suffer from physical injury associated with moving or exchanging tanks. Therefore, there is a need to overcome and/or address one or more of these shortcomings for using exogenously produced NO gas for treating wounds, such as DFU.

### SUMMARY

According to some embodiments of the present disclosure, a nitric oxide (NO) generation system is provided. The NO generation system may include a first reservoir configured to contain a first solution. The first solution may include a nitrite source. The NO generation system may include a first flow generator. The first flow generator may be configured to be in fluid communication with the first reservoir. The NO generation system may include a second reservoir configured to contain a second solution. The second solution may include an acidic solution. The NO generation system may include a second flow generator. The second flow generator may be configured to be in fluid communication with the second reservoir. The NO generation system may include a housing configured to contain a reaction chamber and a gas chamber. The gas chamber may be separated from the reaction chamber by a gas-permeable membrane. The reaction chamber may be disposed downstream of and in fluid communication with at least one of the first flow generator and the second flow generator. For example, the reaction chamber may be in fluid communication with the first reservoir and the second reservoir via the first flow generator and the second flow generator, respectively. The NO generation system may include a carrier gas source disposed upstream of and in fluid communication with the gas chamber.

According to some embodiments of the present disclosure, a nitric oxide (NO) generation system is provided. The NO generation system may include a first reservoir configured to contain a first solution. The first solution may include a nitrite source. The NO generation system may include a first flow generator. The first flow generator may be configured to be in fluid communication with the first reservoir. The NO generation system may include a second reservoir configured to contain a second solution. The second solution may include an acidic solution. The NO generation system may include a second flow generator. The second flow generator may be configured to be in fluid communication with the second reservoir. The NO generation system may include a housing configured to contain a reaction chamber and a gas chamber. The gas chamber may be configured to receive gas from the reaction chamber. The reaction chamber may be configured to contain a liquid. The reaction chamber may be configured to be disposed downstream of and in fluid communication with at least one of the first flow generator and the second flow generator. For example, the reaction chamber may be in fluid communication with the first reservoir and the second reservoir via the first flow generator and the second flow generator, respectively. The NO generation system may include a carrier gas source disposed upstream of and in fluid communication with the reaction chamber or the gas chamber.

According to some embodiments of the present disclosure, a nitric oxide (NO) generation system is provided. The NO generation system may include a first reservoir configured to contain a first solution. The first solution may include a nitrite source. The NO generation system may include a second reservoir configured to contain a second solution. The second solution may include an acidic solution. The NO generation system may include a flow generator. The flow generator may be configured to be in fluid communication with at least one of the first reservoir and the second reservoir. The NO generation system may include a housing configured to contain a reaction chamber and a gas chamber. The gas chamber may be separated from the reaction chamber by a gas-permeable membrane. The reaction chamber may be disposed downstream of and in fluid communication with the flow generator. The NO generation system may include a carrier gas source disposed upstream of and in fluid communication with the gas chamber.

According to some embodiments of the present disclosure, a nitric oxide (NO) generation system is provided. The NO generation system may include a first reservoir configured to contain a first solution. The NO generation system may include a second reservoir configured to contain a second solution. The first solution may include a nitrite source. The second solution may include an acidic solution. The NO generation system may include a first flow generator. The first flow generator may include the first reservoir and may be configured to create a flow of the first solution out of the first reservoir. The NO generation system may include a second flow generator. The second flow generator may include the second reservoir and may be configured to create a flow of the second solution out of the second reservoir. The NO generation system may include a housing configured to contain a reaction chamber and a gas chamber. The gas chamber may be configured to receive gas from the reaction chamber. The reaction chamber may be configured to contain a liquid and may be disposed downstream of and in fluid communication with at least one of the first flow generator and the second flow generator. For example, the reaction chamber may be in fluid communication with the first reservoir and the second reservoir via the first flow generator and the second flow generator, respectively. The NO generation system may include a carrier gas source disposed upstream of and in fluid communication with the reaction chamber or the gas chamber.

In some embodiments, the NO generation system may include a liquid separation tank disposed downstream of and in fluid communication with the reaction chamber. The liquid separation tank may include a first outlet and a second outlet. The NO generation system may include a first circulation circuit disposed downstream of and in fluid communication with the first outlet of the liquid separation tank and disposed upstream of and in fluid communication with the reaction chamber. The NO generation system may include a waste storage unit disposed downstream of and in fluid communication with the second outlet of the liquid separation tank.

In some embodiments, the NO generation system may include a treatment module disposed downstream of and in fluid communication with the gas chamber of the housing. The treatment module may include a treatment device. The treatment device may include at least one of a treatment applicator and a treatment chamber. The treatment applicator may be configured to deliver NO gas to a treatment site. The treatment chamber configured to contain the product gas and cover at least a portion of a treatment site. The NO generation system, such as the treatment module, may include a gas treatment unit disposed downstream of the treatment chamber and configured to absorb NO gas. The NO generation system, such as the treatment module, may include a second circulation circuit configured to generate a fluid flow, such as a product gas flow, relative to the treatment chamber.

According to an embodiment of the present disclosure, a nitric oxide (NO) generation method is provided. The method may include conveying, by a first flow generator, a first solution from a first reservoir to a reaction chamber. The first solution may include a nitrite source. The method may include conveying, by a second flow generator, a second solution from a second reservoir to the reaction chamber. The second solution may include an acidic solution. The method may include mixing, in the reaction chamber, the first solution and the second solution to generate NO gas. For example, the method may include conveying the first solution and the second solution to the reaction chamber, allowing the two solutions to mix in the reaction chamber. Alternatively, the method may include conveying one of the first solution and the second solution to the reaction chamber to mix with the other one that is in the reaction chamber. The method may include transporting NO gas from the reaction chamber to the gas chamber. The method may include transporting, by a gas-permeable membrane, NO gas from the reaction chamber to a gas chamber. The gas-permeable membrane may be configured to separate the gas chamber and the reaction chamber. The method may include transporting, by at least one sparger, NO gas from the reaction chamber to the gas chamber. The method may include conveying, by a carrier gas, the separated NO gas out of the gas chamber. For example, the method may include conveying the separated NO gas from the gas chamber to a treatment module.

It is to be understood that both the foregoing general description and the following detailed description are examples and explanatory only and are not restrictive of the disclosed embodiments as claimed.

The accompanying drawings constitute a part of this specification. The drawings illustrate several embodiments of the present disclosure and, together with the description, serve to explain the principles of certain disclosed embodiments as set forth in the accompanying claims.

### BRIEF DESCRIPTION OF THE DRAWINGS

FIG. 1 is a schematic representation of an NO gas generation system, according to some embodiments of the present disclosure.

FIG. 2A is a schematic representation of an NO gas generation system, according to some embodiments of the present disclosure.

FIG. 2B is a schematic representation of an NO gas generation system, according to some embodiments of the present disclosure.

FIG. 2C is a perspective view of a separation device, according to some embodiments of the present disclosure.

FIG. 2D is a cross-sectional view of the separation device of FIG. 2C.

FIG. 3A is a graphical representation of an NO gas generation system, according to some embodiments of the present disclosure.

FIG. 3B is a cross-sectional view of an applicator of a treatment device, according to some embodiments of the present disclosure.

FIG. 4A is a graphical representation of an NO gas generation system, according to some embodiments of the present disclosure.

FIG. 4B is a graphical representation of an NO gas generation system, according to some embodiments of the present disclosure.

FIG. 5A is a schematic representation of units of an integrated tank in communication with a reaction module of an NO gas generation system, according to some embodiments of the present disclosure.

FIG. 5B is a perspective view of an integrated tank, according to some embodiments of the present disclosure.

FIG. 5C is a partial view of the integrated tank of FIG. 5B.

FIG. 5D is perspective view of the integrated tank of FIG. 5B.

FIG. 5E is a partial perspective view of the integrated tank of FIG. 5B.

FIG. 5F is a partial perspective view of the integrated tank of FIG. 5B.

FIG. 5G is a partial perspective view of the integrated tank of FIG. 5B.

FIG. 5H is a perspective view of the integrated tank of FIG. 5B.

FIG. 5I is a partial perspective view of the integrated tank of FIG. 5B.

FIG. 5J is a partial front view of the integrated tank of FIG. 5B.

FIG. 5K is a front view of the integrated tank of FIG. 5B.

FIG. 6 is a schematic representation of an adaptive control module of an NO gas generation system for controlling NO gas generation, according to some embodiments of the present disclosure.

FIG. 7 is a schematic representation of an NO gas generation process, according to some embodiments of the present disclosure.

FIG. 8 is a graphical representation of NO gas concentration over time during an NO gas generation process, according to some embodiments of the present disclosure.

FIG. 9 is a graphical representation of NO gas concentration over time during an NO gas generation process, according to some embodiments of the present disclosure.

### DETAILED DESCRIPTION

Reference will now be made in detail to disclosed embodiments. The same reference numbers are used throughout the disclosure and figures to reference like components and features. Unless otherwise defined, technical or scientific terms have the meaning commonly understood by one of ordinary skill in the art. It is to be understood that other embodiments may be utilized and that changes may be made without departing from the scope of the disclosed embodiments. Thus, the materials, methods, and examples are illustrative only and are not intended to be necessarily limiting.

The present disclosure provides apparatuses, systems, and methods for generating NO gas from one or more chemical reactions. According to one aspect of the present disclosure, embodiments may output a product gas that includes NO gas. The NO gas in the product gas may be generated or delivered at a predetermined concentration and/or flow rate. For example, a predetermined concentration and/or flow rate of NO gas may be a clinically relevant concentration and/or flow rate of NO gas for treating wounds. A predetermined concentration and/or flow rate of NO gas may be set or selected based on the treatment needed and may be set or selected by a user of embodiments of the present disclosure or a patient. The concentration and/or flow rate of NO gas in the product gas may be adjusted. The generated NO gas may be delivered to a treatment site, such as a would site, an infected site, or a DFU (Diabetic Foot Ulcer) site, and may be delivered in a controlled manner.

The dimensionless unit “ppm” used in the present disclosure to describe gas concentrations refers to parts per million by volume and can be converted to other concentration units, such as parts per million by molar or milligrams per liter (mg/L). The dimensionless unit “%” or “% by volume” used in the present disclosure to describe gas concentrations refers to volume percentage and can be converted to other concentration units, such as weight percentage or molar concentration. As used herein, “about” in a numerical range indicates that the numerical range encompasses normal industry and subject matter variances or tolerances for manufacturing and/or operation. As used herein, the phrase “less than,” “more than,” “between one value and another value,” or “from one value to another value” in a numerical range includes the endpoints and all values within or between the endpoints.

According to an aspect of the present disclosure, some embodiments may allow for NO gas generation over a treatment session that may include at least one operating period. The concentration and/or flow rate of NO gas in the product gas during an operating period may reach and/or remain at a steady state. As described herein, the concentration and/or flow rate of NO gas in the product gas at the steady state may vary from a certain value or a certain range by a steady state error. The steady state error may range from about 0 to about 10%, for example.

According to another aspect of the present disclosure, embodiments may allow for NO gas generation over a treatment session that may include at least one ramp period. As described herein, a ramp period may refer to a transient period during which NO gas concentration of the product gas may increase or decrease from an initial concentration to a steady state concentration. The ramp period may be a ramp-up period or a ramp-down period. The ramp period may be predetermined, adjusted, or minimized to allow for more rapid or immediate provision of a steady stream of NO gas.

According to another aspect of the present disclosure, some embodiments may allow for NO gas generation over a plurality of treatment sessions. The plurality of treatment sessions of NO gas generation may provide NO gas to treat the same patient over time or to treat different patients. One or more parameters for generating or delivering NO gas by some embodiments of the present disclosure may be predetermined and/or adjusted. For example, the number of treatment sessions, the start and end times of a treatment session, the duration of an operating period of a treatment session, the number of operating periods in a treatment session, and/or the concentration and/or flow rate of NO gas in the product gas in one or more operating periods of a treatment session may be predetermined and/or adjusted.

According to another aspect of the present disclosure, some embodiments may allow for NO gas delivery to a wound site at clinically relevant concentrations and/or flow rates for treating wounds. To reduce exposure to health risks, some embodiments may reduce or remove one or more toxic impurities, such as NO₂, that may be present in the product gas before or after NO gas delivery to the wound site.

Various apparatuses, systems, and methods for generating NO gas consistent with the present disclosure are described below.

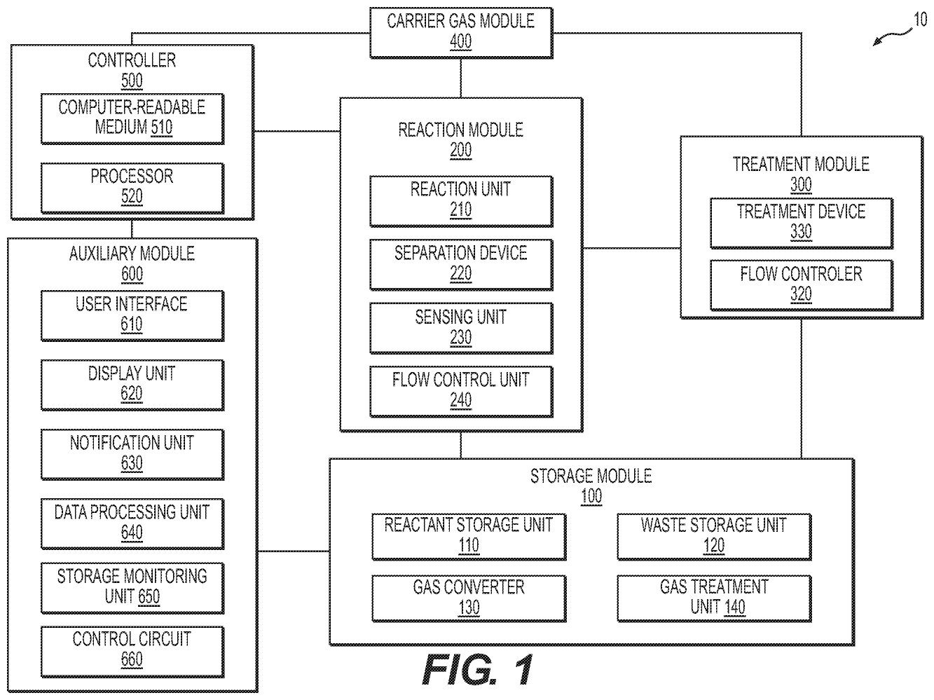

FIG. 1 is a schematic representation of an NO gas generation system 10, according to some embodiments of the present disclosure. NO gas generation system 10 is configured to generate NO gas and control output and/or delivery of the generated NO gas. In some embodiments, system 10 includes a storage module 100 and a reaction module 200. Reaction module 200 may be disposed downstream of and in fluid communication with at least one or more units of storage module 100. In some embodiments, storage module 100 includes a reactant storage unit 110. Reactant storage unit 110 may include one or more reservoirs configured to hold one or more solutions containing reactants for generating NO gas.

In some embodiments, reaction module 200 includes a reaction unit 210. In some embodiments, reaction unit 210 includes a housing configured to contain a reaction chamber and a gas chamber. One or more solutions of reactant storage unit 110 may be transported to the reaction chamber of reaction module 200 to generate NO gas. Two or more solutions may be transported to and mix in the reaction chamber and reactants in the mixed solutions may react and generate NO gas. The gas chamber may be separated from the reaction chamber for transporting the generated NO gas output from reaction module 200. The generated NO gas may diffuse from a reaction chamber into a gas chamber of reaction module 200. For example, NO gas may diffuse from the reaction chamber to the gas chamber through a gas-permeable membrane. The gas-permeable membrane may be permeable to one or more gases, such as NO gas. A product gas containing generated NO gas may be conveyed from the gas chamber of reaction module 200.

In some embodiments, reaction module 200 includes a reaction chamber that may include a liquid region and a gas region. The liquid region may be configured to receive one or more solutions from reactant storage unit 110. The gas region may be configured to receive gas generated in and/or transported from the liquid region. In some embodiments, reaction module 200 includes one or more spargers that may be disposed at any suitable place in the liquid region. The one or more spargers may receive a carrier gas and emanate bubbles of the carrier gas to transport, such as sweep, purge, and/or entrain, NO gas out of the solution in the liquid region. Some examples of the reaction chamber that includes one or more spargers can be found in PCT/CN2021/139117, which is incorporated herein by reference.

In some embodiments, reaction module 200 includes a separation device 220. Separation device 220 is disposed downstream of and in fluid communication with at least one reaction chamber of reaction module 200. In some embodiments, separation device 220 includes a liquid separation tank. Separation device 220 may receive a mixed solution from a reaction chamber of reaction module 200 and separate at least a portion of the mixed solution for recirculation with respect to reaction module 200 as described below.

In some embodiments, reaction module 200 includes a sensing unit 230. Sensing unit 230 includes one or more sensors for detecting parameters of product gas conveyed from reaction module 200, such as a temperature, humidity, flow rate, an NO gas concentration, and/or an NO₂ gas concentration. In some embodiments, the one or more sensors are disposed in an outlet circuit disposed downstream of and in fluid communication with at least one gas chamber of reaction module 200 as described below.

In some embodiments, reaction module 200 includes a flow control unit 240. Flow control unit 240 may include one or more flow generators, such as one or more pumps or syringes. For example, a flow generator may be disposed downstream of and in fluid communication with a reservoir of storage module 100 to transport a solution from the reservoir to reaction module 200. In some embodiments, a flow generator may be disposed downstream of and in fluid communication with two or more reservoirs of storage module 100 and configured to transport a mixed solution from the two or more reservoirs to reaction module 200. In some embodiments, one or more flow generators may be disposed upstream of storage module 100 or may be integrated with storage module 100. For example, the flow generators may be syringe pumps that may provide the one or more reservoirs of storage module 100 as well as allow for transporting the one or more solutions in the reservoirs to reaction module 200.

In some embodiments, a flow generator may include a fluid pump configured to create a fluid flow of a solution at a suitable flow rate. The fluid pump may be any suitable type of fluid pump, such as a syringe pump, a peristaltic pump, a diaphragm pump, or a centrifugal pump. The flow generator may allow for adjusting the flow rate, thereby adjusting the amount of reaction in reaction module 200 and thus NO gas concentration in the product gas of reaction module 200. The flow generator may include a processor in communication with a fluid pump. As described herein, components of system 10 may be in communication with each other via wired or wireless connection. The processor may send fluid control signals to the fluid pump to adjust the flow rate of the fluid pump. The processor may generate the fluid control signals in accordance with instruction signals from a central controller (e.g., controller 500 described below). The flow generator may include a receiver circuit that receives the control signals.

In some embodiments, storage module 100 includes a waste storage unit 120. Waste storage unit 120 is disposed downstream of and in fluid communication with at least one reaction chamber of reaction module 200. In some embodiments, waste storage unit 120 is configured to receive and store solutions and/or gas from reaction module 200 as described below.

In some embodiments, storage module 100 includes a gas converter 130. Gas converter 130 may be disposed downstream of and in fluid communication with at least one gas chamber of reaction module 200. In some embodiments, gas converter 130 is configured to reduce and/or remove one or more impurities in the product gas from reaction module 200 as the product gas passes through gas converter 130.

In some embodiments, system 10 includes a treatment module 300. Treatment module 300 is disposed downstream of and in fluid communication with reaction module 200. For example, treatment module 300 may be disposed downstream of and in fluid communication with at least one gas chamber of reaction module 200. The product gas including NO gas output from reaction module 200 can be conveyed to treatment module 300 to deliver the NO gas to a treatment site. For example, treatment module 300 may include a treatment device 330 configured to deliver NO gas to a treatment site. A treatment site may be a wound site or an infected site, such as a DFU (Diabetic Foot Ulcer) site.

In some embodiments, system 10 includes a gas treatment unit 140. Gas treatment unit 140 may be disposed downstream of and in fluid communication with treatment device 330. In some embodiments, gas treatment unit 140 is configured to filter a waste gas received from treatment module 300 before discharging it to the atmosphere. In some embodiments, storage module 100 includes a gas treatment unit 140 for receiving the waste gas from treatment device 300. Additionally, or alternatively, treatment module 300 includes a gas treatment unit 140 for treating the waste gas.

In some embodiments, treatment module 300 includes a flow controller 320. Flow controller 320 is configured to control and/or monitor flow rate and/or NO gas concentration of the product gas delivered by treatment device 330. As described herein, a flow controller of the present disclosure may include a flow control valve (e.g., an on-and-off valve) or a flow regulating valve configured to regulate a fluid flow. A flow control valve or a flow regulating valve may be opened or closed to pass or stop a fluid flow and/or may also be adjusted to increase or decrease the flow rate of a fluid flow. A flow controller may include a gas sensor. The gas sensor may be configured to detect one or more gases of the product gas delivered by treatment device. For example, the gas sensor may include one or more gas sensors configured to detect an NO gas concentration and/or an NO₂ gas concentration of the product gas to be delivered.

In some embodiments, system 10 includes a carrier gas module 400. Carrier gas module 400 is disposed upstream of and in fluid communication with reaction module 200. Carrier gas module 400 is configured to transport a carrier gas to reaction module 200 to convey generated NO gas out of reaction module 200. For example, the carrier gas may sweep, purge, and/or entrain generated NO gas from at least one gas chamber or a liquid region of the reaction chamber of reaction module 200. The carrier gas may include any suitable gas, such as air, nitrogen, helium, argon, and oxygen. In some embodiments, the carrier gas include ambient air or compressed air.

As shown in FIG. 1, in some embodiments, system 10 includes a controller 500 that controls various operations of system 10. Controller 500 may include a computer-readable medium 510 and a processor 520. Computer-readable medium 510 may be a non-transitory computer-readable medium. Computer-readable medium 510 may store instructions, when executed by processor 520, cause processor 520 to perform operations to control system 10. Processor 520 may include one or more integrated circuits. Controller 500 may be in communication with one or more modules or components of system 10 via a wired or wireless connection. In some embodiments, controller 500 is in communication with storage module 100, reaction module 200, and carrier gas module 400 to control NO gas generation, such as the flow rate and/or concentration of generated NO gas. In some embodiments, controller 500 is in communication with various sensors and/or control units of system to monitor and/or control one or more conditions or processes of system 10 as described below.

In some embodiments, system 10 may include an auxiliary module 600 configured to provide various functions. Auxiliary module 600 may include one or more of a group of functional units including a user interface 610, a display unit 620, a notification unit 630, a data processing unit 640, and a storage monitoring unit 650. Auxiliary module 600 may include a control circuit 660 connected to one or more of the functional units. Control circuit 660 may include an integrated circuit (IC) panel and/or a control bus. Auxiliary module 600 may be in communication with one or more modules or components of system 10, such as storage module 100, reaction module 200, treatment module 300, carrier gas module 400, and controller 500, and may send and/or receive data from these modules. Various embodiments of auxiliary module 600 are described below.

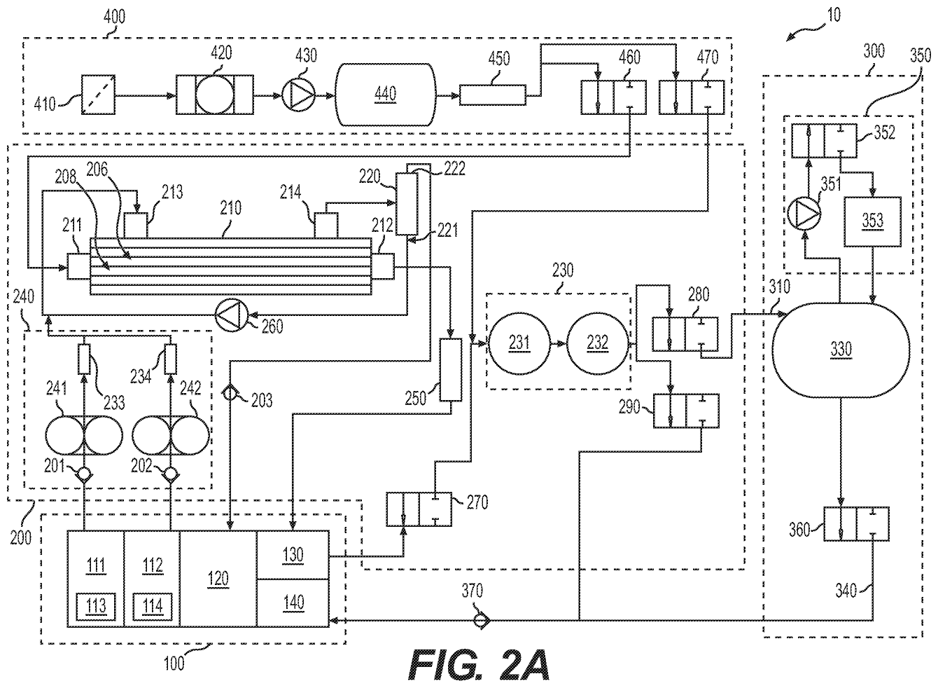

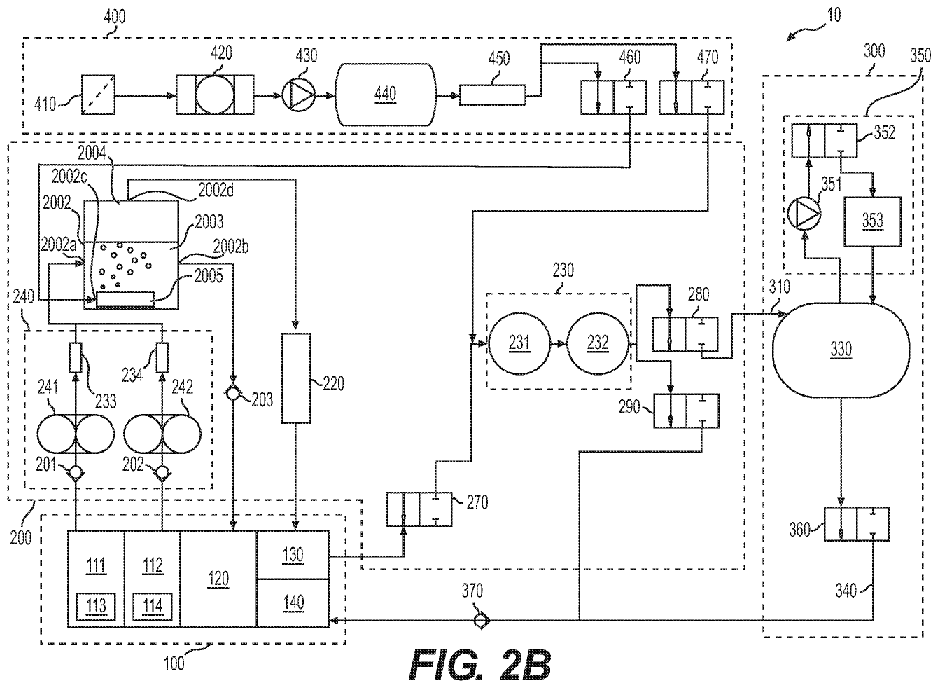

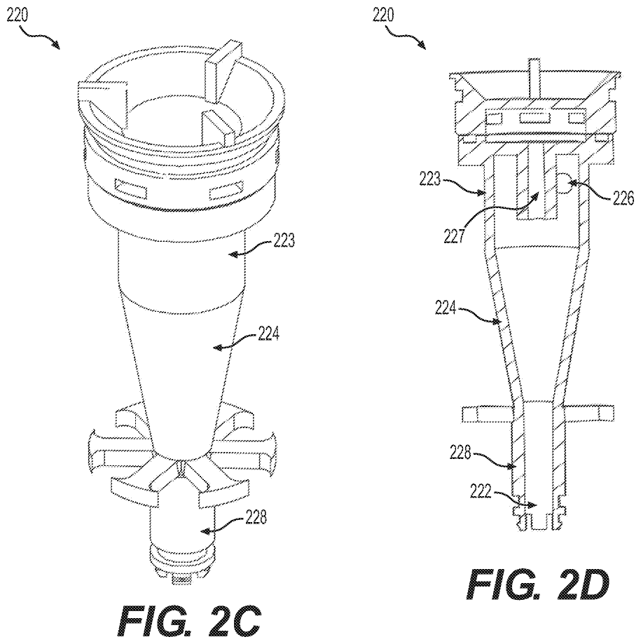

FIG. 2A is a schematic representation of an NO gas generation system 10, according to some embodiments of the present disclosure. In some embodiments, reactant storage unit 110 of reaction module 200 may include one or more reservoirs for storing one or more solutions for generating NO gas. For example, reactant storage unit 110 may include a first reservoir 111 and a second reservoir 112. First reservoir 111 is configured to contain a solution A and the second reservoir 112 is configured to contain a solution B. In some embodiments, solution A includes a source of nitrite ions. In some embodiments, solution B includes an acidic solution, such as an acid solution or an acidic buffer solution. When solutions A and B are mixed together, in some embodiments, the nitrite ions in solution A may react with hydrogen ions in solution B and generate NO gas. In some embodiments, reactant storage unit 110 include one or more additional reservoirs for storing additional amounts of solutions A and/or B, or other solutions that may facilitate NO gas generation.

The source of nitrite ions in solution A may include one or more nitrite salts. The nitrite salt may be an organic nitrite salt or an inorganic nitrite salt. Examples of organic nitrite salts include organic ammonium nitrite salts, such as tetramethylammonium nitrite and tetraethylammonium nitrite. Examples of inorganic nitrite salts include metal nitrite salts, such as nitrite salts of Li, Na, K, Rb, Ca, Mg, Al, and Fe. Some other examples of the source of nitrite ions can be found in PCT/US2018/027081 and PCT/CN2021/139117. PCT/US2018/027081 and PCT/CN2021/139117 are incorporated herein by reference. The concentration of a nitrite salt in solution A may range from about 1.0 wt % to about 10.0 wt %, for example.

In some embodiments, the one or more reducing agents in solution B include one or more acids. In some embodiments, the acid may be a strong acid, such as hydrochloric acid, sulfuric acid, etc. In some embodiments, the acid may be a weak acid, such as ascorbic acid (also known as Vitamin C or L-ascorbic acid), acetic acid, citric acid, oxalic acid, maleic acid (also known as apple acid or L-maleic acid), etc. In some embodiments, the one or more reducing agents in solution B include one or more buffering agents. For example, the buffering agent may be sodium citrate, sodium acetate, disodium phosphate, sodium dihydrogen phosphate, glycine, potassium hydrogen phthalate, HEPES (4-(2-hydroxyethyl)-1-piperazineethanesulfonic acid), sodium bicarbonate, sodium carbonate, etc. The pH value of solution B may be less than 6.5. For example, the pH value of solution B may be equal to or less than 3. In some embodiments, reducing the pH or increasing the concentration of the acid or hydrogen ions in solution B may increase the NO gas concentration in the product gas generated from mixing solutions A and B as illustrated in Examples A-C below.

In some embodiments, reactant storage unit 110 includes one or more sensors configured to detect one or more conditions of one or more solutions in reactant storage unit 110. The one or more conditions may include temperature, volume, flow rate, and reactant concentration. A first temperature sensor 113 may be disposed in or adjacent first reservoir 111 for detecting the temperature of solution A. A second temperature sensor 114 may be disposed in or adjacent second reservoir 112 for detecting the temperature of solution B. Any suitable temperature sensors may be used, such semiconductor-based sensors, infrared sensors, thermistors, thermocouples, etc. In some embodiments, a pressure sensor may be disposed in or adjacent a reservoir and the volume of the solution in the reservoir may be obtained based on the pressure value detected by the pressure sensor. In some embodiments, a liquid level sensor, such as an infrared sensor, an ultrasound sensor, or a capacitive sensor, may be disposed in or adjacent a reservoir and configured to measure the liquid level in the reservoir. The liquid level may be used to determine the volume of solution in the reservoir. In some embodiments, the one or more sensors are in communication with storage monitoring unit 650 and/or controller 500 (see FIG. 1) to send signals indicating the detected conditions to storage monitoring unit 650 and/or controller 500. Storage monitoring unit 650 may record the identification of the solution and the detected conditions based on the signals from the one or more sensors.

In some embodiments, reaction unit 210 of reaction module 200 includes a housing that contains one or more gas chambers 206 and one or more reaction chambers 208. Reaction chambers 208 may be disposed downstream of and in fluid communication with one or more reservoirs of reactant storage unit 110. A flow generator of flow control unit 240 may create a fluid flow from a reservoir of reactant storage unit 110 to a reaction chamber of reaction module 200. In some embodiments, reaction chamber 208 may be disposed downstream of and in fluid communication with both first reservoir 111 and second reservoir 112.

In some embodiments, a flow generator 241 is disposed downstream of and in fluid communication with first reservoir 111 and configured to create a flow of solution A from first reservoir 111 to reaction chamber 208. In some embodiments, a flow generator 242 is disposed downstream of and in fluid communication with second reservoir 112 and is configured to create a flow of solution B from second reservoir 112 to the same reaction chamber to mix with solution A in the reaction chamber. In some embodiments, flow generator 241 may be disposed downstream of and in fluid communication with both reservoirs 111 and 112 of storage module 100 and configured to transport a mixed solution from the reservoirs to reaction module 200. In such instances, flow generator 242 may be omitted. As discussed above, a flow generator may include a suitable fluid pump configured to create a fluid flow of a solution at a suitable flow rate. In some embodiments, a flow generator may be integrated with a reservoir of storage module 100. For example, instead of being disposed downstream of a reservoir, a flow generator, such as flow generator 241 or 242, may include a syringe pump that includes a reservoir for storing a solution, a piston, and a driver that moves the piston to deliver the solution in the reservoir to the reaction chamber. Controller 500 may control and/or adjust the flow rates of one or more of the flow generators.

In some embodiments, flow control unit 240 includes one or more flow controllers disposed downstream of reactant storage unit 110 to regulate the flow of the solutions from reactant storage unit 110. In some embodiments, flow control unit 240 includes a first flow controller 201 disposed downstream of reservoir 111 and upstream of reaction chamber 208 of reaction module 200. First flow controller 201 may be disposed upstream of or downstream of flow generator 241. In some embodiments, flow control unit 240 includes a second flow controller 202 disposed downstream of reservoir 112 and upstream of reaction chamber 208. Flow controller 202 may be disposed downstream of or upstream of flow generator 242. In some embodiments, at least one of first flow controller 201 and second flow controller 202 may include a one-way valve configured to prevent the solutions from flowing back to reactant storage unit 110 from the reaction chamber of reactant storage unit 110.

In some embodiments, as shown in FIG. 2A, flow control unit 240 includes one or more sensors, such as a first sensor 233 and a second sensor 234. In some embodiments, the sensors include flow sensors configured to detect the flow rates of one or more solutions from reactant storage unit 110 to reaction chamber 208. As described herein, a flow sensor may also be referred to as a flow meter that can measure a flow rate of a liquid or a gas flow. The flow rate may be a linear, nonlinear, mass, or volumetric flow rate. For example, first sensor 233 may include a flow sensor disposed downstream of reservoir 111 and/or downstream of flow generator 241. Second sensor 234 may include a flow sensor disposed downstream of reservoir 112 and/or downstream of flow generator 242. In some embodiments, the sensors include temperature sensors configured to detect the temperature of the fluid flowing from reactant storage unit 110 to reaction chamber 208. For example, in addition to or in placement of using first temperature sensor 113 and second temperature sensor 114, first sensor 233 and second sensor 234 may each include a temperature sensor that detect the temperatures of the solutions flowing by.

In some embodiments, sensors 233 and 234 are in communication with storage monitoring unit 650 and/or controller 500 (see FIG. 1) to send signals indicating the measured conditions of the solutions, such as flow rates and temperatures, to storage monitoring unit 650 and/or controller 500. In some embodiments, storage monitoring unit 650 may record the identification of a solution and the measured conditions based on the received signals. In some embodiments, storage monitoring unit 650 may determine the remaining volume of at least one solution in reactant storage unit 110 based on the initial volume of the solution and the flow rate of the solution detected by a flow sensor. In some embodiments, controller 500 may adjust the flow rate of at least one solution from reactant storage unit 110 based on a predetermined NO gas concentration or concentration range as described below.

In some embodiments, a gas chamber 206 is separated from an adjacent reaction chamber 208 in the housing of reaction unit 210 by one or more gas-permeable membranes disposed in the housing of reaction unit 210. The gas-permeable membranes may include one or more materials configured to allow gas molecules, such as NO gas molecules, to pass through from the reaction chamber to the gas chamber. The material of the gas-permeable membrane may have a porous structure having an average pore size or a pore size distribution that allows gas molecules, such as NO gas molecules, to pass through. The material of the gas-permeable membrane may include one or more of polydimethylsiloxane (PDMS), silicone, polypropylene, and poly (4-methyl-1-pentene), for example.

In some embodiments, gas chamber 206 of reaction unit 210 includes a gas inlet 211 and a gas outlet 212 and reaction chamber 208 includes a liquid inlet 213 and a liquid outlet 214. Solutions A and B may enter reaction chamber 208 via liquid inlet 213 and then mix and react in the reaction chamber to generate NO gas. The generated NO gas may diffuse from reaction chamber 208 to at least one adjacent gas chamber 206. The gas chamber, reaction chamber, and the gas-permeable membrane may have any suitable configuration. For example, the housing of reaction unit 210 may enclose a plurality of hollow fibers formed by a gas-permeable membrane. In some instances, each hollow fiber may have an inlet and an outlet that are in fluid communication with gas inlet 211 and gas outlet 212 respectively such that the space in the hollow fiber constitutes a gas chamber. In other instances, each hollow fiber may have an inlet and outlet that are in fluid communication with liquid inlet 213 and liquid outlet 214 respectively such that the space in the hollow fiber constitutes a reaction chamber.

In some embodiments, reaction module 200 includes a separation device 220 disposed downstream of reaction unit 210. Separation device 220 is disposed downstream of and in fluid communication with one or more reaction chambers 208 of reaction unit 210, such as via liquid outlet 214. In some embodiments, separation device 220 includes a first outlet 221 and a second outlet 222. In some embodiments, separation device 220 is configured to receive a liquid, such as a mixed solution of solutions A and B, from reaction unit 210, such as a reaction chamber 208, and separate a portion of the received liquid to outlet 221 and another portion of the received liquid and/or some residual gas from the reaction chamber to outlet 222.

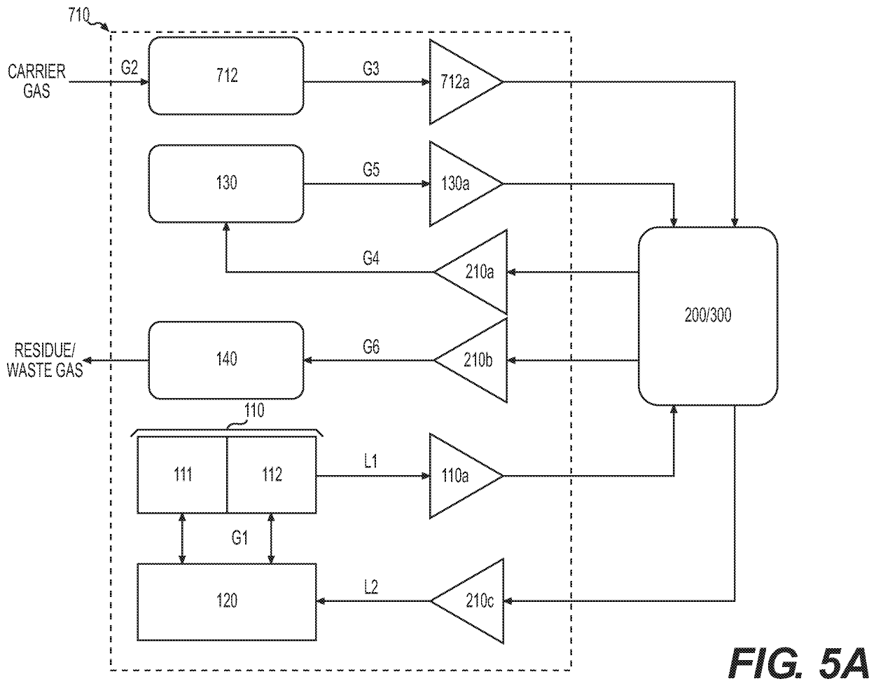

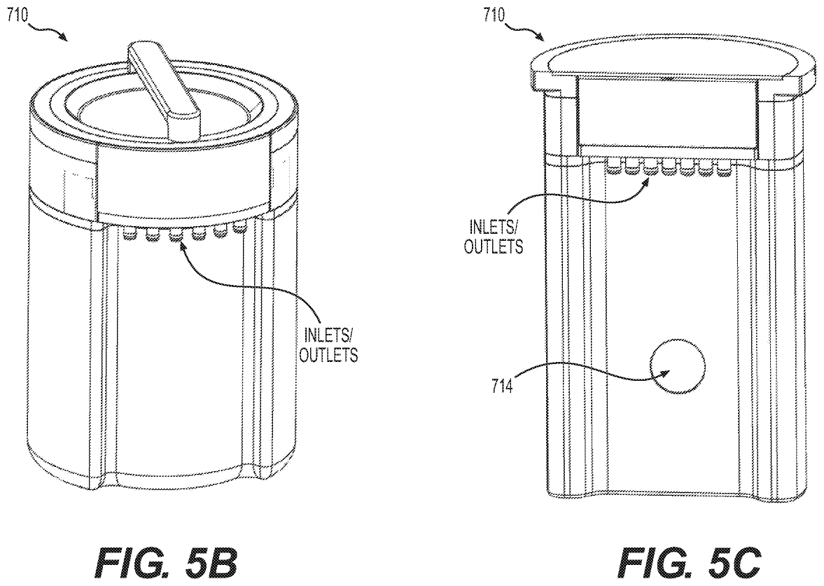

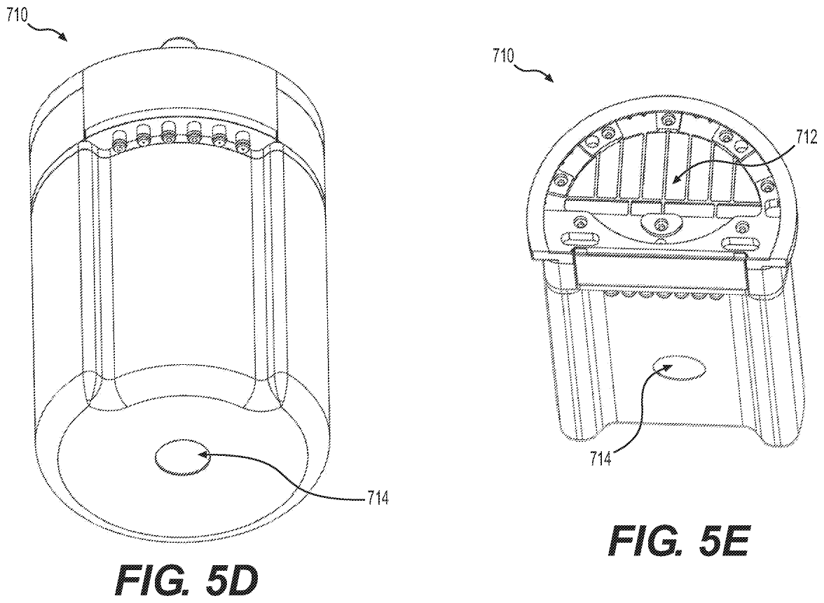

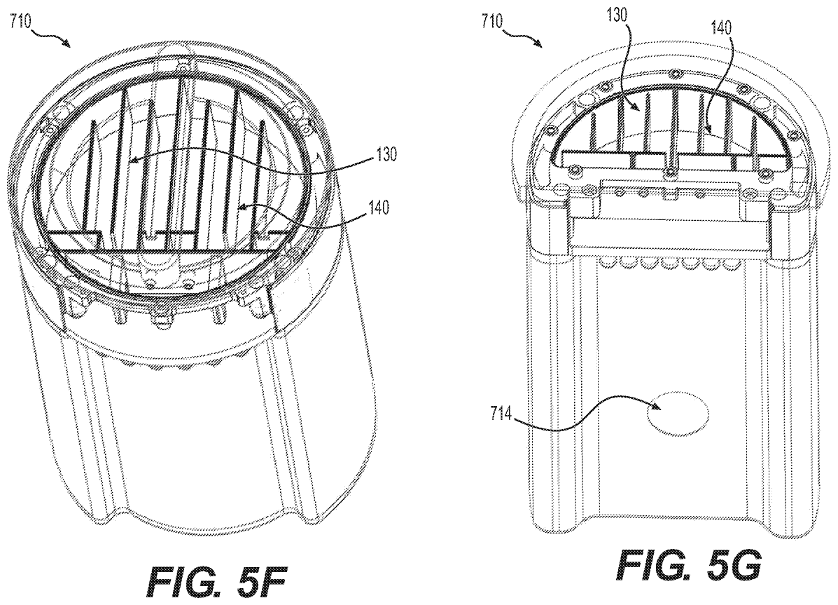

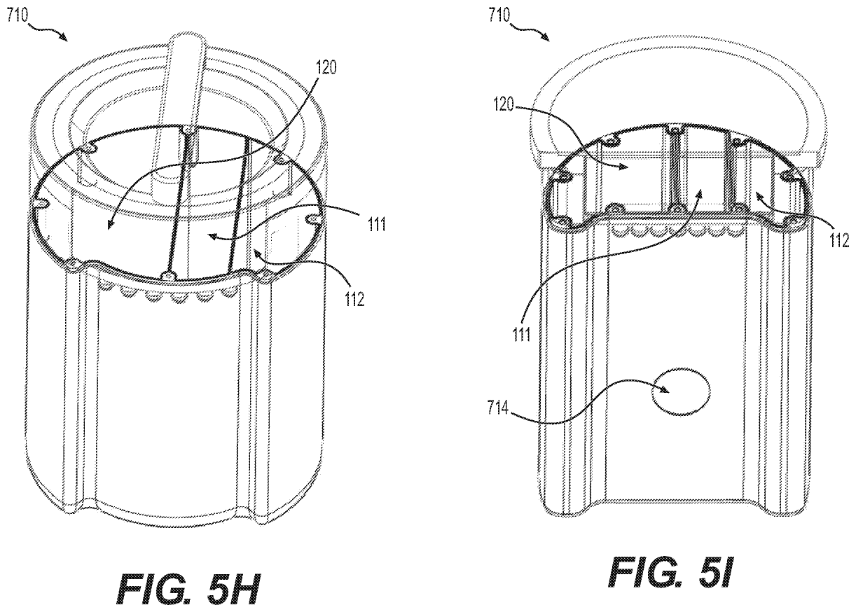

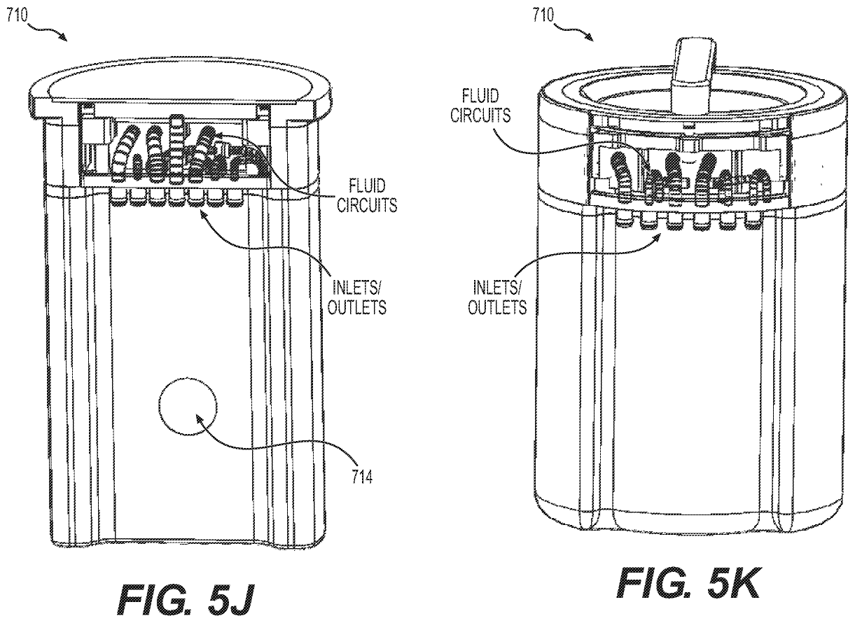

In some embodiments, separation device 220 includes a liquid separation tank. The liquid separation tank may have a cylindrical shape disposed along a longitudinal axis. The liquid separation tank may have one or more chambers that have inlets and outlets in fluid communication to allow liquid in a fluid flow including both liquid and gas to settle out and separate from the gas in the fluid flow. Exemplary structures of the liquid separation tank can be found in the description of embodiments of filtration device 508 shown in FIGS. 5A-5C in PCT/CN2021/139117. For example, the liquid settled out from the fluid flow may be accumulated in a buffer chamber, which may include outlet 221 to transport the accumulated liquid back to the reaction chamber.

FIGS. 2C-2D illustrate another embodiment of separation device 220. As shown in FIGS. 2C-2D, in some embodiments, separation device 220 includes a cyclone separator configured to separate gas from liquid in a fluid flow containing liquid and gas by way of centrifugal force. In some embodiments, the cyclone separator includes a body portion 223 and a cyclone portion 224. Body portion 223 may have a cylindrical shape. In some embodiments, body portion 223 includes an inlet 226 and a first outlet channel 227. A fluid flow containing liquid and gas from the reaction chamber may enter the cyclone separator via inlet 226. Cyclone portion 224 may have an inverted cone shape configured to create a spiral vortex of the fluid flow received by the cyclone separator. The gas in the fluid flow having less inertia may travel up to outlet channel 227 due to the vortex while the liquid in the fluid flow having more inertial may be forced to separate from the gas and drop down. In some embodiments, the cyclone separator includes a collection portion 228 configured to collect and discharge the liquid separated from the gas. In some embodiments, collection portion 228 includes liquid outlet 222 for discharging the separated liquid. The separated liquid may be circulated back to the reaction chamber of reaction module 200 via the liquid circulation circuit.

In some embodiments, reaction module 200 includes a liquid circulation circuit. In some embodiments, the liquid circulation circuit includes a circulation inlet disposed downstream of and in fluid communication with outlet 221 of separation device 220. In some embodiments, the liquid circulation circuit includes a circulation outlet in fluid communication with liquid inlet 213. In some embodiments, the liquid circulation circuit includes a flow generator 260 configured to generate a recirculated liquid flow relative to reaction unit 210. Flow generator 260 may be a fluid pump, such as a peristaltic pump, a diaphragm pump, a circulating pump, etc. In some embodiments, the liquid circulation circuit may circulate a mixed solution of solutions A and B from outlet 221 of separation device 220 relative to at least one reaction chamber 208 of reaction unit 210. In some embodiments, the recirculated mixed solution may be combined with a flow of solution A and/or a flow of solution B and enter the reaction chamber of reaction unit 210. The recirculation of the mixed solution may facilitate mixing of the solutions in reaction chamber 208 of reaction unit 210, may reduce the time needed to generate a steady supply of NO gas, and/or may increase the NO gas concentration in the product gas of reaction module 200.

In some embodiments, second outlet 222 of separation device 220 is disposed upstream of and in fluid communication with waste storage unit 120 of storage module 100. In some instances, at least a portion of NO gas generated in the reaction chamber of reaction unit 210 may not diffuse to the gas chamber and may remain in the reaction chamber. In some embodiments, separation device 220 is configured to receive a mixture of NO gas and liquid (e.g., a mixed solution of solutions A and B) from the reaction chamber and to allow separation of at least a portion of the liquid from the gas based on, for example, gravity settling or segregation. For example, a separated portion of liquid may exist from outlet 221 and be recirculated to reaction unit 210. A remaining portion of the liquid and NO gas from the reaction chamber may exist from outlet 222. The liquid and gas existing outlet 222 may be transported to waste storage unit 120 in storage module 100. The liquid and/or gas in waste storage unit 120 may be reused and/or recycled. In some embodiments, a flow controller 203 may be disposed upstream of waste storage unit 120 and downstream of separation device 220. Flow controller 203 may be a one-way valve configured to prevent backward flow of the liquid and gas transported to and/or stored in waste storage unit 120.

In some embodiments, NO gas generated in reaction module 200 may be conveyed out of reaction module 200 by a carrier gas received from carrier gas module 400. For example, carrier gas module 400 may be disposed upstream of and in fluid communication with gas chamber 206 of reaction unit 210. In some embodiments, carrier gas module 400 may generate a supply of carrier gas and operate as a carrier gas source itself. In some embodiments, carrier gas module 400 may receive a supply of carrier gas from a carrier gas source disposed upstream of carrier gas module 400 and transport a carrier gas flow to reaction unit 210. The carrier gas may include any suitable gas, such as air, nitrogen, helium, and argon. In some embodiments, carrier gas module 400 generate a carrier gas flow from ambient air or compressed air.

In some embodiments, carrier gas module 400 may include an inlet circuit disposed upstream of and in fluid communication with gas inlet 211 of reaction unit 210. The inlet circuit may be configured to generate and convey a carrier gas flow into gas chamber 206. The inlet circuit of carrier gas module 400 may include a flow generator 430. Flow generator 430 may be in fluid communication with a carrier gas source, such as ambient air or compressed air, and drive a carrier gas flow in the inlet circuit. Flow generator 430 may include a vacuum pump, a diaphragm pump, a turbo pump, a rotary vane pump, a piston pump, a screw pump, a peristaltic pump, or any other suitable pump.

In some embodiments, the inlet circuit of carrier gas module 400 may include a filtration unit disposed upstream of and in fluid communication with gas inlet 211 of reaction unit 210. The filtration unit may include one or more filters configured to reduce or eliminate one or more impurities of a carrier gas entering or passing through the inlet circuit. For example, the filtration unit may include a primary filter 410 and a secondary filter 420. Primary filter 410 may reduce or remove solid matter from the carrier gas flowing therethrough. Secondary filter 420 may reduce or remove moisture from the carrier gas flowing therethrough.

In some embodiments, carrier gas module 400 may include a gas capacitor 440 disposed downstream of and in fluid communication with flow generator 430. Gas capacitor 440 may receive and store the carrier gas to stabilize the flow rate of the carrier gas. This may allow for providing a carrier gas flow with a steady flow rate to reaction unit 210.

In some embodiments, the inlet circuit of carrier gas module 400 includes one or more flow control devices to control the carrier gas flow in the inlet circuit and/or entering reaction unit 210. Carrier gas module 400 may include a flow sensor 450 disposed downstream of gas capacitor 440. Flow sensor 450 is configured to detect a flow rate (e.g., mass or volumetric flow rate) of a carrier gas flow from gas capacitor 440. In some embodiments, carrier gas module 400 may include a flow controller 460 disposed upstream of reaction unit 210 to regulate the carrier gas flow from the inlet circuit to reaction unit 210. Flow controller 460 may be disposed downstream of and in communication with flow sensor 450 to adjust the flow rate of the carrier gas according to measurement of flow sensor 450.

In some embodiments, reaction module 200 includes an outlet circuit configured to convey NO gas generated in reaction unit 210. In some embodiments, the outlet circuit is disposed downstream of and in fluid communication with gas outlet 212 of reaction unit 210. In some embodiments, the outlet circuit is disposed upstream of and in fluid communication with a treatment module 300 of system 10. In some embodiments, the outlet circuit configured to convey a product gas containing NO gas generated in reaction unit 210 to treatment module 300.

In some embodiments, carrier gas module 400 further includes a flow controller 470 that may control transporting a carrier gas flow directly to the outlet circuit of reaction module 200. Flow controller 470 may be disposed downstream of and in communication with flow sensor 450 to adjust the flow rate of the carrier gas according to measurement of flow sensor 450. In some embodiments, flow controller 470 is disposed upstream of an NO gas sensor 231 of the outlet circuit of reaction module 200. The carrier gas flow conveyed directly to the outlet circuit may purge the outlet circuit to prepare for NO generation or may be combined with a product gas containing NO gas output from reaction unit 210 to dilute the NO gas to suitable treatment concentration if needed. In some embodiments, the carrier gas flow can be further conveyed to treatment device 330 via flow controller 470 to supply gas to treatment device 330 or to purge a treatment chamber, inlet circuit, and/or outlet circuit of treatment device 330.

Reaction module 200 may include one or more filtration systems or devices to reduce or remove one or more impurities in the product gas. In some embodiments, the outlet circuit of reaction module 200 includes a filtration device 250 disposed downstream of and in fluid communication with gas chamber 206 of reaction unit 210, for example, via gas outlet 212. The filtration device may reduce or remove liquid and/or solid matter from a product gas containing NO gas conveyed from the gas chamber.

In some embodiments, filtration device 250 includes one or more moisture filters. The moisture filter may reduce or remove liquid, such as water, in the vapor phase and/or the liquid phase. As described herein, moisture may include any liquid, in vapor phase or in liquid phase, that may be present in the product gas, such as water vapor, water droplets, solvent vapor, and solvent droplets. In some embodiments, the moisture filter includes a membrane filter. In some embodiments, the membrane filter includes a polymeric material. The polymeric material may have a porous structure. The polymeric material may absorb liquid vapor and/or liquid droplets. Additionally, or alternatively, the polymeric material may be at least partially impermeable to liquid vapor and/or liquid droplets. For example, the membrane filter may include a Nafion™ membrane.

In some embodiments, filtration device 250 includes one or more solid matter filters. The solid matter filter may be configured to filter any type of solid matter by, for example, modifying or selecting the filter material and/or pore size. In some embodiments, the solid filter may be a salt aerosol filter. In some embodiments, the solid matter filter includes a membrane filter. The membrane filter may include a polymeric material that has a porous structure. For example, the polymeric material may include one or more selected from polytetrafluoroethylene (PTFE), polyvinylidene fluoride, polyethersulfone, mixed cellulose ester, polyamide (nylon), nylon 6, and nylon 66. Some examples of the solid matter filter can be found in PCT/CN2021/139117.

In some embodiments, the outlet circuit of reaction module 200 includes gas converter 130. Gas converter 130 may be disposed downstream of and in fluid communication with gas outlet 212 of reaction unit 210. In some embodiments, gas converter 130 is disposed downstream of filtration device 250. Gas converter 130 and filtration device 250 may be separate components or may be integrated into one component. Gas converter 130 may configured to reduce one or more impurities in the product gas as the product gas passes therethrough. NO gas may be oxidized to one or more toxic nitrogen oxides during generation and/or transportation, such as NO₂, which may impose health risks if delivered with NO gas to treat patient and leaked to the ambient air. Gas converter 130 may absorb some or all potential toxic nitrogen oxides, such as NO₂, that may be present in the product gas to NO gas as the product gas passes through it. Gas converter 130 may reduce the potential risk of exposure to toxic nitrogen oxides.

Gas converter 130 may increase NO gas concentration in the product gas by converting one or more other nitrogen oxides in the product gas back to NO gas. In some embodiments, gas converter 130 may convert some or all potential toxic nitrogen oxides, such as NO₂, that may be present in the product gas to NO gas as the product gas passes through it.

In some embodiments, gas converter 130 includes a body, an inlet, and an outlet. The inlet and the outlet are in fluid communication with a cavity defined by the body. At least a portion of the cavity is filled with a filter material that may absorb or convert one or more toxic nitrogen oxides, such as NO₂, as the product gas passes through the filter material. Some examples of the gas converter 130 can be found in PCT/CN2021/139117 with reference to the description of embodiments of gas converter 800.

In some embodiments, reaction module 200 includes a sensing unit 230 configured to detect one or more conditions of the product gas from reaction unit 210. In some embodiments, sensing unit 230 includes a flow sensor to detect the flow rate of the product gas. In some embodiments, sensing unit 230 includes a moisture sensor to detect the moisture level of the product gas. In some embodiments, sensing unit 230 includes one or more gas sensors to detect a concentration of one or more gases in the product gas. In some embodiments, sensing unit 230 includes an NO gas sensor 231 configured to detect a NO gas concentration of the product gas. In some embodiments, sensing unit 230 includes an NO₂ sensor 232 configured to detect a NO₂ gas concentration of the product gas.

In some embodiments, sensing unit 230 includes a temperature sensor and/or a humidity sensor. The measurements of the temperature sensor and/or humidity sensor may be used for calibrating the measurements of one or more gas sensors of sensing unit 230. In some embodiments, reaction module 200 includes a humidifier disposed upstream of sensing unit 230 to adjust moisture of the product gas passing through sensing unit 230. Adjusting the humidity of the product gas may improve the accuracy of one or more of the gas sensors of sensing unit 230.

In some embodiments, one or more sensors of sensing unit 230 may be calibrated periodically, on an as-needed basis, or prior to generating or delivering NO gas. For example, reaction module 200 may include a calibration circuit (not shown) disposed upstream of and in fluid communication with sensing unit 230 to transport a calibration gas to one or more sensors of sensing unit 230. The calibration gas may include a standard gas having known concentrations of O₂, NO, and/or NO₂. One or more sensors of sensing unit 230 may measure one or more gas concentrations of the calibration gas as the calibration gas purges or passes through sensing unit 230 and may be calibrated based on the measurements. In some embodiments, the calibration gas circuit is in fluid communication with carrier gas module 400. For example, the calibration gas may be the carrier gas from carrier gas module 400. A flow controller may control the calibration gas flow in the calibration gas circuit. The flow controller may open the circuit during calibration measurements and may close the calibration gas circuit after the calibration measurements.

In some embodiments, sensing unit 230 or a sensor of sensing unit 230 may be disposed at any suitable location that allows the sensor to be disposed in contact with the product gas of reaction unit 210. In some embodiments, a sensor is disposed in or adjacent the outlet circuit of reaction module 200. In some embodiments, sensing unit 230 is disposed downstream of the outlet circuit or downstream of one or more filters or filtration devices of the outlet circuit. Sensing unit 230 is disposed downstream of filtration device 250 and/or gas converter 130 to detect one or more conditions of the product gas to be transported to treatment module 300. In some embodiments, NO₂ sensor 232 of sensing unit 230 is disposed downstream of NO gas sensor 231.

In some embodiments, the outlet circuit of reaction module 200 includes one or more flow controllers configured to control the flow rate and/or direction of the product gas flow in the outlet circuit. In some embodiments, the outlet circuit may include a first flow controller 270 disposed downstream of gas converter 130. In some embodiments, the outlet circuit may include a second flow controller 280 disposed downstream of gas converter 130 and sensing unit 230. In some embodiments, the outlet circuit may include a third flow controller 290 disposed downstream of gas converter 130. Some functions of these flow controllers are described below.

In some embodiments, flow controller 270 may be closed to stop product flow from gas converter 130, and flow controllers 470 and 280 may be opened to allow the carrier gas to flow into the outlet circuit of reaction module 200 to be conveyed to treatment module 300. The carrier gas conveyed to treatment module 300 without NO gas may be used to remove, purge, or replace residue gas in treatment module 300, such as in a treatment chamber of treatment module 300, before or after a treatment session.

In some embodiments, second flow controller 280 may be closed to stop product gas flow to treatment module 300, and third flow controller 290 may open to allow residual product gas in the outlet circuit of reaction module 200 to be conveyed to gas treatment unit 140. In such instances, flow controller 270 may open to allow residual product gas to be transported from reaction unit 210 to gas treatment unit 140. Additionally, or alternatively, first controller 270 and flow controller 460 may close, and flow controller 470 may open to allow the carrier gas to sweep and transport residual product gas in the outlet circuit to gas treatment unit 140.

In some embodiments, treatment module 300 is disposed downstream of and in fluid communication with the outlet circuit of reaction module 200. In some embodiments, treatment module 300 includes an inlet circuit 310 and treatment device 330. In some embodiments, inlet circuit 310 is disposed downstream of and in fluid communication with the outlet circuit of reaction module 200 and treatment device 330. The product gas containing NO gas from reaction module 200 is conveyed to treatment device 330 via the outlet circuit of reaction module 200 and inlet circuit 310 of treatment module 300. In some embodiments, a flow controller 280 is disposed upstream of inlet circuit 310 to regulate the product gas flow entering inlet circuit 310 and/or treatment device 330.

In some embodiments, treatment device 330 includes a treatment applicator, such as a portable applicator. The treatment applicator may be configured to deliver NO gas to a treatment site. In some embodiments, the treatment applicator may include flow controller 320 configured to control and/or monitor the flow rate of the gas flow delivered by the applicator. In some embodiments, flow controller 320 may include a flow control valve and/or a flow sensor. In some embodiments, the flow control valve is an on and off valve. In some embodiments, the treatment applicator may include one or more gas sensors, such as an NO gas sensor and an NO₂ gas sensor, that monitor the concentration of NO gas and/or concentration of NO₂ gas in the output gas flow of the applicator. In some embodiments, the treatment applicator may include an indicator, such as a display and its associate circuit, for displaying the measured gas concentration and/or flow rate to the user of treatment device 330.

In some embodiments, treatment device 330 includes a treatment chamber. A treatment chamber may be configured to surround, enclose, or immerse at least a portion of a treatment site. For example, a treatment chamber may be provided with an at least partially closed container, such as a bag, a box, a cover, a hood, a housing, or other similar containers. The container may have an opening for receiving or covering a treatment site or a body portion having the treatment site. In some embodiments, at least a portion of the container is transparent or translucent such that the treatment site is visible from outside of the container. In some embodiments, the container may be reused or disposed after one or more uses.

In some embodiments, the treatment chamber of treatment device 330 is configured to accumulate the product gas and/or the NO gas in the product gas in the treatment chamber. In some embodiments, treatment module 300 includes a gas circulation circuit 350 configured to generate a recirculated gas flow relative to the treatment chamber. In some embodiments, gas circulation circuit 350 includes a flow generator 351, a flow controller 352, and a gas filter 353. In some embodiments, flow generator 351 is a gas pump. Flow generator 351 may recirculate a flow of the product gas and/or NO gas relative to treatment chamber. Flow controller 352 may be disposed downstream of flow generator 351. Flow controller 352 may regulate the circulated gas flow and may detect a flow rate and NO gas concentration of the recirculated gas flow. In some embodiments, flow controller 352 includes a flow control valve and/or a gas sensor. The flow control valve may control a flow rate of the recirculated product gas flow. The gas sensor may detect an NO gas concentration of the recirculated product gas flow. Gas filter 353 may be disposed downstream of flow controller 352 and may be disposed upstream of the treatment chamber. In some embodiments, gas filter 353 absorbs or converts NO₂ gas that may be present in the recirculated product gas as the recirculated product gas passes therethrough. Gas filter 353 may have the same or similar functions and/or structural configurations as gas converter 130.

In some embodiments, treatment module 300 includes a recovery circuit 340 disposed downstream of and in fluid communication with the treatment chamber and upstream of and in fluid communication with gas treatment unit 140. Recovery circuit 340 may transport waste gas from the treatment chamber to gas treatment unit 140. In some embodiments, storage module 100 includes gas treatment unit 140. In some embodiments, treatment module 300 includes gas treatment unit 140. In some embodiments, both storage module 100 and treatment module 300 includes a gas treatment unit 140. In some embodiments, recovery circuit 340 includes a flow controller 360 disposed downstream of the treatment chamber and upstream of gas treatment unit 140. In some embodiments, flow controller 360 is a one-way valve configured to prevent gas flow from gas treatment unit 140 to the treatment chamber. In some embodiments, waste gas from treatment module 300 may pass through gas treatment unit 140 before being discharged, such as to the atmosphere.

Waste gas from treatment module 300 may include one or more components, such as NO gas, the carrier gas, moisture, and/or other nitrogen oxides that may be generated during NO gas generation, transportation, and/or delivery. Nitrogen oxides (also referred to as NOx), such as NO and NO₂, may contribute to air pollution and/or pose health risks if directly released from system 10 to the atmosphere. In some embodiments, gas treatment unit 140 is configured to reduce or eliminate one or more nitrogen oxides, such as NO and NO₂, in the waste gas from treatment module 300 before discharging it to the atmosphere, thereby reducing or eliminating potential air pollution and/or risk of exposure to nitrogen oxides. In some embodiments, gas treatment unit 140 includes a plurality of baffles configured to define a circuitous flow path. In some embodiments, at least a portion of the flow path is filled with a filter material configured to react with and/or absorb one or more nitrogen oxides, such as NO gas and NO₂ gas, in the waste gas flow as it passes through the filter material. Some examples of the structure and/or filter material of gas treatment unit 140 can be found in the description of embodiments of waste gas treatment device 700 in PCT/CN2021/139117.

FIG. 2B is a schematic representation of NO gas generation system 10, according to some embodiments of the present disclosure. As shown in FIG. 2B, in some embodiments, reaction unit 210 of reaction module 200 includes a housing 2002 including a liquid region 2003 and a gas region 2004. Liquid region 2003 may be a liquid chamber configured to contain liquid. Gas region 2004 may be a gas chamber configured to contain gas and/or receive gas from liquid region 2003 or the liquid chamber. In some embodiments, housing 2002 includes a liquid inlet 2002a and a liquid outlet 2002b both in fluid communication with liquid region 2003. In some embodiments, liquid region 2003 is disposed downstream of and in fluid communication with one or more reservoirs of reactant storage unit 110. One or more flow generators, such as flow generators 241, 242, may transport one or more solutions, such as solutions A and B, from reactant storage unit 110 to liquid region 2003 via liquid inlet 2002a. The one or more solutions may mix in liquid region 2003 of housing 2002 and generate NO gas. The solution in housing 2002 may be transported out of housing 2002 via liquid outlet 2002b. Liquid outlet 2002b may be disposed upstream of and in fluid communication with waste storage unit 120. The solution in housing 2002 may be transported to waste storage unit 120 after entering and/or mixing in housing 2002 for a period of time. In some embodiments, the solution in housing 2002 may be recirculated (see FIG. 2A) via a liquid circulation circuit before being transported to waste storage unit 120.

In some embodiments, gas region 2004 of housing 2002 is configured to receive NO gas generated in and/or transported from liquid region 2003. In some embodiments, housing 2002 includes a gas inlet 2002c and a gas outlet 2002d. Gas inlet 2002c may be in fluid communication with liquid region 2003 and configured to receive a carrier gas from carrier gas module 400. The carrier gas may be conveyed into the solution in liquid region 2003. For example, the carrier gas may sweep, purge, and/or entrain generated NO gas from the solution in liquid region 2003 to gas region 2004. Gas outlet 2002d may be in fluid communication with gas region 2004, and NO gas accumulated in gas region 2004 may be conveyed or transported out of housing 2002 by the carrier gas via gas outlet 2002d.

In some embodiments, as shown in FIG. 2B, one or more spargers 2005 are disposed in liquid region 2003. Sparger 2005 may be in fluid communication with gas inlet 2002c and configured to generate bubbles from a carrier gas received via gas inlet 2002c. As used herein, a sparger may include a device or system configured to emanate gas bubbles into a liquid. A sparger may also be referred to as a bubbler. The one or more spargers 2005 may be disposed at any suitable place in liquid region 2003 to emanate bubbles of the carrier gas to transport, such as sweep, purge, and/or entrain, NO gas out of the solution in liquid region 2003. Exemplary structures and/or configurations of a sparger can be found in the description of embodiments of sparger 134 in PCT/CN2021/139117.

In some embodiments, separation device 220 is disposed downstream of and in fluid communication with gas region 2004. Separation device 220 may receive a fluid flow from gas region 2004 containing both gas and liquid (e.g., liquid droplets). Separation device 220 may separate at least a portion of the liquid from the gas in the fluid flow. The separated liquid may be collected in separation device 220. The separated gas may be conveyed from separation device 220 to gas converter 130 disposed downstream of separation device 220.

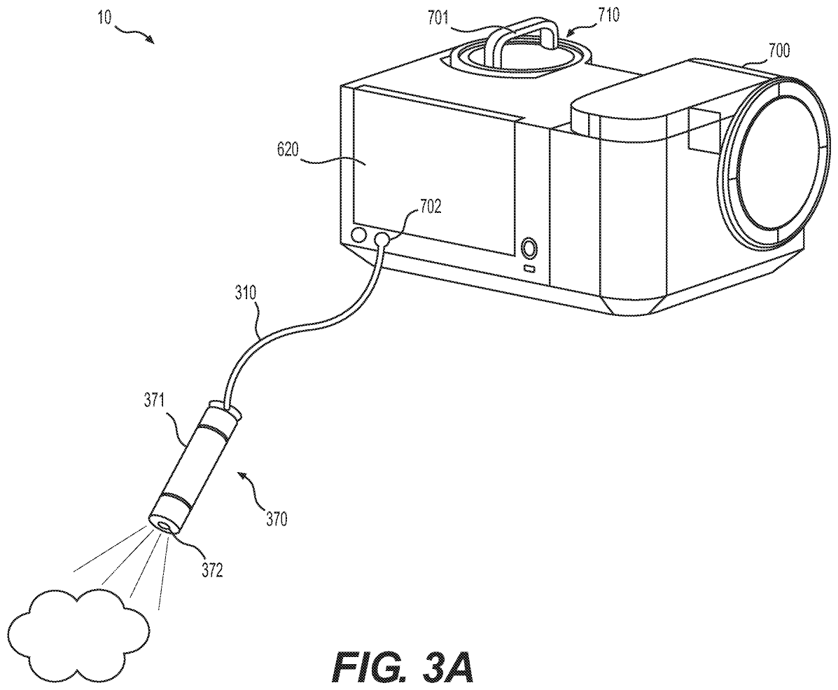

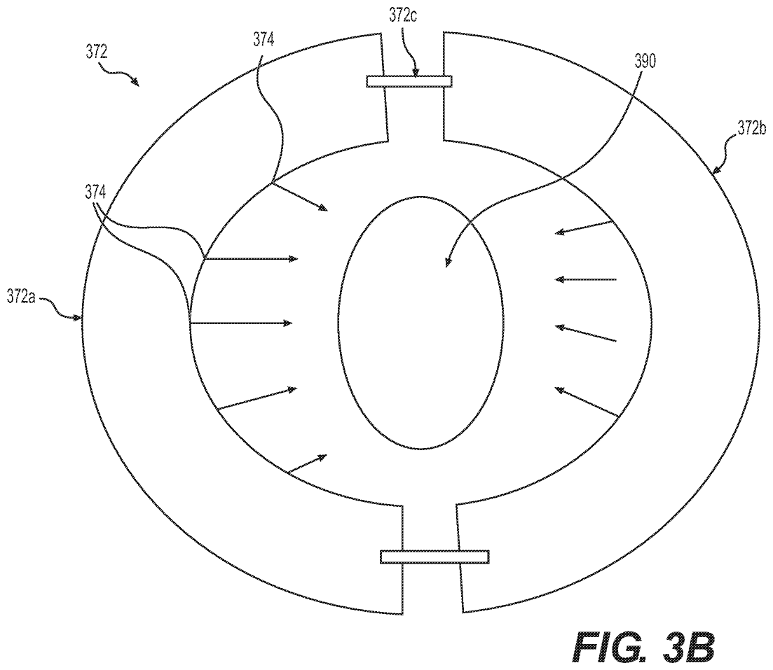

FIG. 3A is a graphical representation of NO gas generation system 10, according to some embodiments of the present disclosure. System 10 includes a housing 700 that encloses various modules of system 10 described above, such as storage module 100, reaction module 200, carrier gas module 400, and controller 500. In some embodiments, system 10 includes an integrated tank 710, at least a portion of which may be received in housing 700. Integrated tank 710 may be accessible via a side surface or a top surface of housing 700. Integrated tank 710 may integrate one or more units or modules of system 10 that use materials that may need to be replenished or replaced, such as reaction storage unit 110, waste storage unit 120, gas converter 130, gas treatment unit 140, and reaction unit 210. Integrated tank 710 may include a cover 701 for accessing the units therein for replenishing and/or replacing materials for NO gas generation and/or delivery. Cover 701 may have a handle for easy removal or accessing the units in integrated tank 710. Embodiments of integrated tank 710 are described below with reference to FIGS. 5A-5K.

In some embodiments, treatment module 300 includes a portable applicator 370. Portable application 370 may be a hand-held applicator. In some embodiments, portable applicator 370 includes a body 371 for being held or fixed at a suitable position. In some embodiments, portable applicator 370 includes an open end 373, such as a nozzle, disposed at a distal end of portable applicator 370 for delivering a product gas flow and/or an NO gas flow to a treatment site. In some embodiments, inlet circuit 310 of treatment module 300 fluidly connects portable applicator 370 with an outlet 702 on housing 700. Outlet 702 may be in fluid communication with reaction module 200, such as the outlet circuit of reaction module 200, for receiving the product gas generated in reaction module 200. Inlet circuit 310 may include a flexible tubing of a suitable length such that portable applicator 370 can be moved close to a treatment site at different locations, which may increase precision of NO gas delivery to the treatment site.

In some embodiments, system 10 includes a power circuit and a power connector or wire projecting out of housing 700 for connecting to a power supply. In some embodiments, housing 700 encloses at least a portion of auxiliary module 600. Auxiliary module 600 is configured to provide various functions for the control and operation of system 10. In some embodiments, as shown in FIG. 3A, auxiliary module 600 includes a user interface 610 in communication with controller 500 of system 10. In some embodiments, user interface 610 includes one or more input/output devices such as, for example, a touchscreen, a keyboard, a mouse, a track pad, a knob, a switch, and the like. User interface 610 may receive instructions from a user to select or set treatment protocols. A treatment protocol may include various treatment parameters, such as a period of a treatment session, a number of treatment sessions, a predetermined concentration and/or flow rate of NO gas in one or more treatment sessions, and the start and end times of a treatment session.

User interface 610 may be used for various functions. For example, user interface 610 may be used to search and export treatment data, calibrate sensors, and/or set system configuration. System configurations may include configuration of one or more flow generators, flow controllers, data processing and/or storage settings, display settings, notification and/or alert settings, etc. User interface 610 also may be used to clear alert or notification information. User interface 610 may be used to control, start, end operations of system 10 and its modules or components, and start system self-trouble shooting and self-checking, etc.

In some embodiments, user interface 610 includes a display unit 620. Display unit 620 may include a display and its driver and power circuits. Display unit 620 may include a screen, a projector, or any other suitable device that can display information to user. In some embodiments, display unit 620 includes a touchscreen. In some embodiments, display unit 620 is configured to display one or more conditions of system 10, such as the temperature, volume, flow rate, and concentration of one or more solutions in storage module 100, and the flow rate, temperature, and/or concentration measured by one or more sensors of system 10. In some embodiments, display unit 620 is configured to display the one or more conditions of system 10 in real time such that the user may monitor the operation of the system and/or may monitor the generation and/or delivery of NO gas in real time. In some embodiments, display unit 620 is configured to display one or more parameters of a treatment protocol selected or set by user. In some embodiments, display unit 620 is configured to display alert information, such as low volumes of solutions in storage module 100, concentrations NO gas and NO₂ gas out of safety or predetermined ranges. Display unit 620 may be configured to display various other information for operating and maintaining system 10, such as calibration information, system logs, self-trouble shooting menu when system 10 is in error, malfunction or fault report of one or more components of system 10, and proposed solutions for addressing system errors or malfunctions.

In some embodiments, auxiliary module 600 may include a notification unit 630 in communication with controller 500. Notification unit 630 may include any suitable device and its driver and power circuits for providing notification to the user of system 10. For example, notification unit 630 may include a display, a light indicator, a speaker, an alarm, or the like. In some embodiments, notification unit 630 is a part of display unit 620. Notification unit 630 may be configured to generate notification before, during, and/or after operation of system 10 for NO gas generation.

In some embodiments, notification unit 630 is configured to generate different alert signals, such as different text displays, various voice or sound alerts, and various colors of lighting, to provide different notifications to the user. For example, notification unit 630 may be configured to generate different alerts for various system malfunctions, errors, or faults, such as system alerts, treatment alerts, and consumable material alerts. System alerts may include sensor fault alerts, abnormal gas transportation alerts, such as when a flow sensor detecting an abnormal flow rate of a gas flow, and abnormal liquid transportation alerts, such as when a flow sensor detecting an abnormal flow rate of a solution. Treatment alerts may include NO gas concentration alerts, such as when a sensor detects an NO gas concentration exceeding a threshold or a predetermined range, and NO₂ gas concentration alerts, such as when a sensor detects an NO₂ gas concentration in the output gas of system or an NO₂ gas concentration in the atmosphere exceeds a threshold or a predetermined range. Consumable material alerts may include low solution supply alerts, solution expiration alerts, and incorrect installation or non-installation of one or more units of the integrated tank.

In some embodiments, auxiliary module 600 may include a data processing unit 640 in communication with controller 500. Data processing unit 640 may include a transceiver circuit and a non-transitory storage device. In some embodiments, data processing unit 640 is configured to receive, transmit, upload, export, and/or print information related to system configuration or information related to treatment protocols or sessions. Data processing unit 640 may send the information to a computing device external to system 10 or to a cloud-based system via wired or wireless communication. Data processing unit 640 may be operated by a user with administrator right of access to system 10.

In some embodiments, auxiliary module 600 may include a storage monitoring unit 650. Storage monitoring unit 650 may include a transceiver circuit and a non-transitory storage device. Storage monitoring unit 650 may be configured to receive and record information relating to the identification and/or one or more conditions of one or more solutions stored in reactant storage unit 110. The one or more conditions of a solution may include temperature, volume, flow rate, expiration date, and concentration of a solution. The information may be provided by the user via user interface 610, received from reactant storage unit 110 (e.g., sensors. labels, or tags), or may be received from an external computing device or a cloud system. For example, reactant storage unit 110 may include one or more Radio Frequency Identification (RFID) labels that include information about unique identifications of the reactant storage unit, the date, time, type, and volume of a solution stored in the reactant storage unit. Storage monitoring unit 650 may include a RFID reader that may acquire such information from reactant storage unit 110 to facilitate management and maintenance of storage module 100. Storage monitoring unit 650 may also send its received or recorded information to display unit 620 for displaying to the user of system 10.

In some embodiments, when the flow rate of one or more solutions received by storage monitoring unit 650 is abnormal, storage monitoring unit 650 may send the abnormal information to controller 500, display unit 620, and/or notification unit 630. An alert signal may be generated, such as by controller 500 or notification unit 630, and sent to a user by notification unit 630. Storage monitoring unit 650 may allow for monitoring the conditions, such as flow rates, temperatures, and volumes (e.g., consumed volume and/or remaining volume), of the solutions in reactant storage unit 110 in real time. Such monitoring of reactant storage unit 110 may allow for adjusting conditions of the solutions based on the measured conditions to adjust the flow rate and/or concentration of generated NO gas and improve treatment efficacy and safety, proper maintenance, such as replenishing or replacing reactant storage unit 110.

In some embodiments, storage monitoring unit 650 is configured to receive and record information relating to one or more conditions of waste liquid and/or gas stored in waste storage unit 120, such as temperature, volume, and pressure. For example, a volume sensor may be configured to detect the volume of liquid stored in waste storage unit 120, and a pressure sensor may be configured to detect a gas pressure in waste storage unit 120. Such monitoring of waste storage unit 120 may allow for timely maintenance of waste storage unit 120 and normal operation of system 10. For example, storage monitoring unit 650 may send the received or recorded information to display unit 620 to display to the user and/or to notification unit 630 to alert the user of abnormal conditions of waste storage unit 120.

In some embodiments, portable applicator 370 includes an annular applicator configured to deliver NO gas to a treatment site. FIG. 3B is a cross-sectional view of an annular applicator 372, according to some embodiments of the present disclosure. Annular applicator 372 may have a ring-shaped structure. In some embodiments, the ring-shaped structure has one-piece, such as a ring. In some embodiments, the ring-shaped structure has a plurality of pieces, such as two half rings 372a and 372b as shown in FIG. 3B. In some embodiments, an inner circumference of annular applicator 372 can be adjusted. Annular applicator 372 may include one or more elastic connections 372c between two or more pieces, such as half rings 372a and 372b. Elastic connection 372c may be an elastic cord, band, strip, or fabric, for example. Elastic connection 372c may have an original length and may be stretched to extend over its original length to increase the inner circumference of annular applicator 372 to adapt to the size of treatment site 390 or a body portion having the treatment site. In some embodiments, an inner volume of annular applicator 372 is in fluid communication with inlet circuit 310 for receiving the product gas containing NO gas. A plurality of outlets 374 are provided on an inner surface of annular applicator 372 that faces treatment side 390 or the body portion having the treatment site. For example, at least one of the inner surfaces of half rings 372a and 372b include one or more outlets 374 configured to output a flow of the product gas delivered to annular applicator 372. The outlets 374 may be distributed according to a pattern and/or density. Different ones of outlets 374 may be opened or closed to make a pattern to deliver NO gas to one or more particular treatment sites. The pattern of the outlets may be symmetric, asymmetric, or random. The pattern and/or density may be pre-configured based on the size, number, and/or type of treatment site 390.

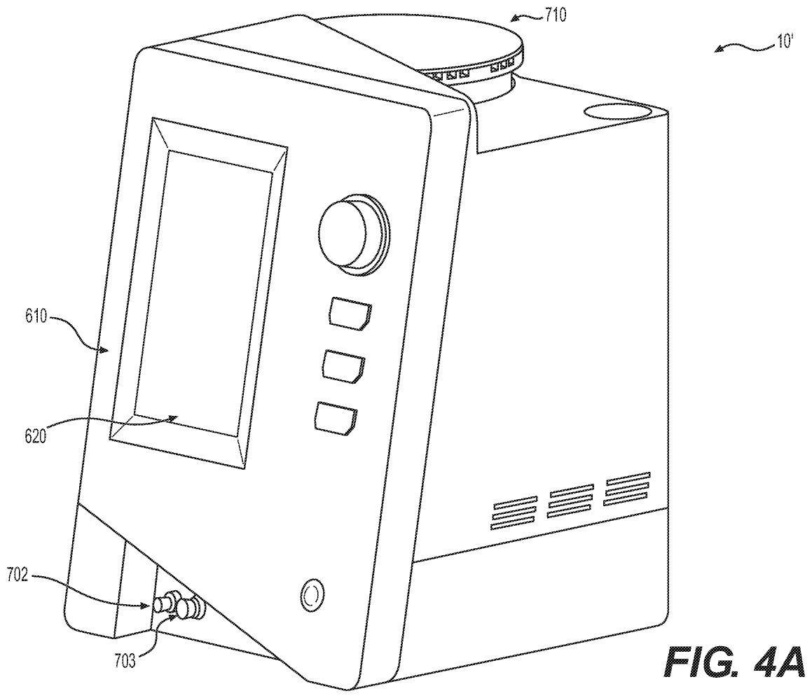

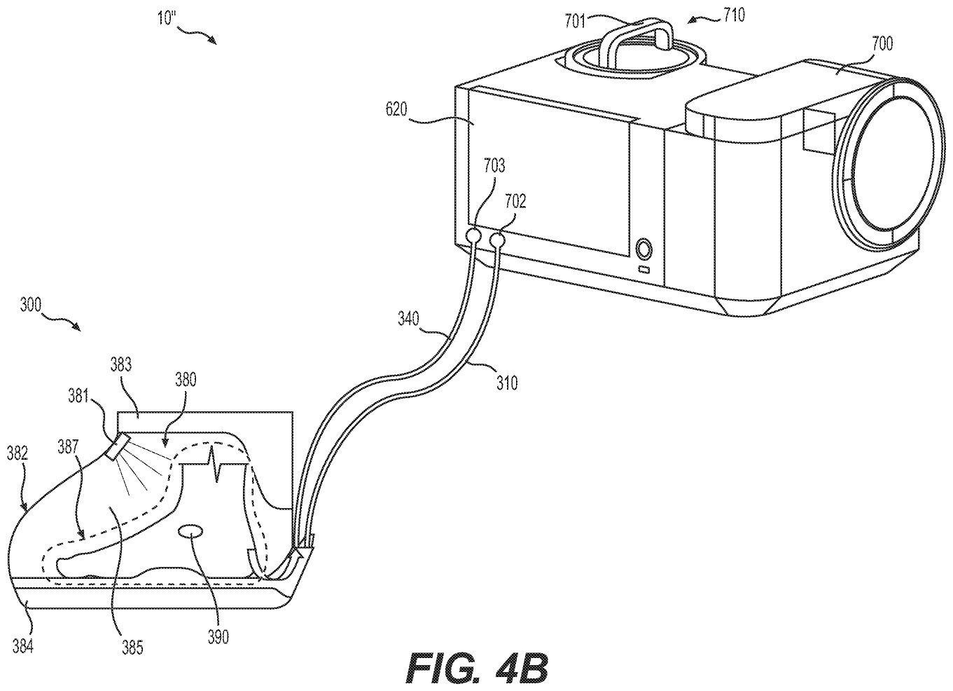

FIG. 4A is a graphical representation of an NO gas generation system 10′, according to some embodiments of the present disclosure. Similar to system 10 as in FIG. 3A, at least a portion of integrated tank 710 may be disposed inside housing 700. System 10′ may have components similar to those described above for system 10 with reference to FIG. 3A, which are not repeated herein. In some embodiments, one or more units or components of auxiliary module 600 of system 10′ are provided on housing 700, such as user interface 610, display unit 620, and notification unit 630.

In some embodiments, system 10′ includes an inlet 703. Inlet 703 may be in fluid communication with recovery circuit 340. In some embodiments, treatment module 300 of system 10′ includes a treatment chamber (not shown) in fluid communication with inlet circuit 310 (see FIG. 2A) and recovery circuit 340. A product gas flow containing NO gas may enter treatment chamber 380 via inlet circuit 310 and recirculated to system 10′ via recovery circuit 340 and inlet 703. The recirculated product gas may be transported to gas treatment unit 140.

FIG. 4B is a graphical representation of an NO gas generation system 10″, according to some embodiments of the present disclosure. Components of system 10″ similar to those described above with reference to FIG. 3A are not repeat herein. In some embodiments, system 10″ includes inlet circuit 310 in fluid communication with outlet 702 on housing 700. In some embodiments, system 10″ includes recovery circuit 340 and inlet 703 in fluid communication with recovery circuit 340. In some embodiments, treatment module 300 of system 10 includes a treatment chamber 380 in fluid communication with inlet circuit 310 and recovery circuit 340. A product gas flow containing NO gas may enter treatment chamber 380 via inlet circuit 310 and recirculated to system 10″ via recovery circuit 340. The recirculated product gas may be circulated with respect to treatment chamber 380 via gas circulation circuit 350 or may be transported to gas treatment unit 140. As described herein, systems 10′ and 10″ may have one or more same or similar components, units, modules, and features, and may perform same or similar processes for NO gas generation as described for system 10 throughout this disclosure. In some embodiments, treatment module 300 may include a cover 382 providing treatment chamber 380. Cover 382 may include a top portion 383, a bottom portion 384, and a body portion 385. Top portion 383 may provide an opening for receiving a treatment site 390, such as a foot with DFU. The opening may be closed to form a seal. Bottom portion 384 may provide a surface for supporting a body part, such as a foot. In some embodiments, bottom portion 384 may provide a surface made of a soft or elastic material to reduce pressure on the body part or treatment site. In some embodiments, body portion 385 is made of a transparent or translucent material to allow the treatment site to be visible to a user or patient. In some embodiments, bottom portion 384 includes a gas converter 130 and/or a gas treatment unit 140. The product gas within the treatment chamber 380 may be treated by gas converter 130 to increase NO gas concentration. Waste gas from treatment chamber 379 may be treated by gas treatment unit 140 before being released to the atmosphere or recirculated back to system 10″.

In some embodiments, treatment chamber 380 is configured for immersing treatment site 390 in the product gas for a treatment session, during which a product gas containing NO gas may be transported to and accumulate in treatment chamber 380. After a treatment session, system 10″ may stop generating and transporting NO gas to treatment chamber 380. System 10″ may then generate and transport a carrier gas flow to treatment chamber 380. The carrier gas flow may replace the accumulated product gas in treatment chamber 380. The replaced gas from treatment chamber 380 may be released to the atmosphere, e.g., after being treated by a gas treatment unit 140 of treatment module 300, or may be recirculated to system 10″, for a period of time or until NO gas concentration in treatment chamber 380 is below a threshold.

In some embodiments, inlet circuit 310 may fluidly connect with treatment chamber 380 at a first connection and recovery circuit 340 may connect with treatment chamber 380 at a second connection. The first connection and the second connection may be designed to be disposed at locations of the treatment chamber to increase distribution of NO gas in the treatment chamber. In some embodiments, the first connection and the second connection are disposed near or adjacent each other. In some embodiments, the first connection and the second connection are disposed apart, for example, at opposite ends, corners, or sides of treatment chamber 380. In some embodiments, treatment chamber 380 further includes a treatment light 381 configured to provide phototherapy to the treatment site before, during, and/or after NO gas treatment.

In some embodiments, a bag 387 may be provided in cover 382. In such instances, the space in bag 387 provides a treatment chamber and the space between bag 387 and cover 382 provides a buffer chamber for receiving waste gas from the treatment chamber in bag 387. Bag 387 may be in fluid communication with inlet circuit 310 to receive product gas. The space between bag 387 and cover 382 may be in fluid communication with recovery circuit 340 to recirculate the waste gas back to system 10″. In some embodiments, treatment module 300 includes a gas treatment unit 140 and the space between bag 387 and cover 382 may be in fluid communication with the gas treatment unit 140 to treat the waste gas before releasing it to the atmosphere.

In some embodiments, treatment module 300 may provide a display module for displaying treatment information, such as a concentration of NO gas in treatment chamber 380 in real time, a change of the concertation of NO gas during a treatment period, etc. In some embodiments, treatment module 300 may provide an alert module for generating an alert or warning message when a condition, such as error or fault, occurs during the treatment period, such as waste gas leakage, high NO₂ gas concentration, low NO gas concentration, low volume of a solution, etc. In some embodiments, treatment module 300 includes a control module (not shown), for example, in top portion 383. The control module may be configured to provide a user interface for setting one or more configurations and/or controlling one or more functionalities of treatment module 300, such as adjusting recirculation flow rate of the product gas in the treatment chamber 380, controlling treatment light 381, etc.

FIGS. 5A to 5K illustrate an integrated tank 710 that may be installed in system 10, according to some embodiments of the present disclosure. Integrated tank 710 may integrate one or more units of system 10. Integrated tank 710 may allow for closed internal circulation of gas and/or liquid in system 10 and reduce or prevent gas or liquid leaking from system 10. The integration of various units in integrated tank 710 may reduce the size and improve portability of system 10 and may allow for easier control and monitor of the integrated units. Integrated tank 710 may be installed in or uninstalled from system 10 as one integrated tank.

FIG. 5A is a schematic representation of units of integrated tank 710 in communication with reaction module 200 and/or treatment module 300, according to some embodiments of the present disclosure. In some embodiments, integrated tank 710 includes one or more components of system 10 that are configured to contain, pass, filter, or treat a fluid, such as gas or liquid, such as reaction storage unit 110 (e.g., first reservoir 111 and second reservoir 112), waste storage unit 120, gas converter 130, and gas treatment unit 140. In some embodiments, integrated tank 710 includes one or more inlets and outlets. The inlets may be gas inlets or liquid inlets. The outlets may be gas outlets or liquid outlet. The inlets and outlets may be in fluid communication with reaction module 200, a source of fluid (e.g., atmosphere or carrier gas module 400), or the atmosphere. A fluid flowing in integrated tank 710 may flow along only one direction. Integrated tank 710 may include one or more flow controllers that controls the direction of a fluid flow through the units in integrated tank 710.

In some embodiments, integrated tank 710 includes one or more gas circuits connecting one or more units for conveying, filtering, or treating a gas flow, and one or more liquid circuits connecting one or more units for storing or conveying a liquid flow. As described herein, a circuit may include one or more conduits for conveying one or more fluid flows. For example, integrated tank 710 may include one or more of gas circuits G1-G6 and one or more of liquid circuits L1-L2. Each circuit may include a flow controller that controls the direction of the fluid flow through the circuit. For example, solutions stored in reactant storage unit 110 (e.g., first reservoir 111 and second reservoir 112) may be transported to reaction module 200 via a liquid circuit L1. Liquid circuit L1 may include two or more conduits for transporting two or more solutions, respectively, and may include one or more flow controllers 110a for controlling the direction and/or flow rate of the fluid flowing in liquid circuit L1.

Waste liquid from reaction module 200 may be transported to waste storage unit 120 via liquid circuit L2, which may include a flow controller 210c. In some instances, some gas may be transported with the waste liquid to waste storage unit 120, such as some residual gas received from outlet 222 of separation device 200. The gas transported to waste storage unit 120 may be stored in waste storage unit 120. During operation, as more solutions in reactant storage unit 110 are used and become waste liquid, some of the gas stored in waste storage unit 120 may be conveyed to one or more reservoirs of reaction storage unit 110 via gas circuit G1, which allows for keeping the gas pressure in waste storage unit 120 and/or other units of integrated tank 710 within a suitable range.

In some embodiments, integrated tank 710 includes a buffer chamber 712. A carrier gas may be first conveyed to buffer chamber 712, and then may be conveyed from buffer chamber 712 to reaction module 200 via gas circuit G3. Gas circuit G3 may include a flow controller 712a. Buffer chamber 712 may include one or more filters or filter materials for reducing or removing one or more impurities in the carrier gas, such as moisture and solid matter. The product gas generated in reaction module 200 may be conveyed to gas converter 130 via gas circuit G4, which may include a flow controller 210a. The product gas treated by gas converter 130 may be conveyed back to reaction module 200 via gas circuit G5. Gas circuit G5 may include a flow controller 130a. Residue gas in reaction module 200 and/or waste gas from treatment module 300 may be conveyed to gas treatment unit 140 via gas circuit G6 and then released to the atmosphere. Gas circuit G6 may include a flow controller 210b.

The various components or units of integrated tank 710 may be arranged in a compact manner. Each component or unit may be sealed from other components, such as via one or more sealing rings, and may be releasably interlocked with one or more adjacent components, such as via nuts and bolts, threading, friction fitting, or snap fit mechanisms.

In some embodiments, the components and units of integrated tank 710 are arranged in one or more vertical layers. For example, FIGS. 5B-5K show a three-layer configuration of the units of integrated tank 710. Integrated tank 710 may include buffer chamber 712 in a top layer (see FIG. 5E), gas converter 130 and gas treatment unit 140 in a middle layer (see FIGS. 5F-5G), and reactant storage unit 110 (e.g., reservoirs 111 and 112) and waste storage unit 120 in a bottom layer (see FIGS. 5H-5I). The middle layer may provide a plurality of baffles. Gas may flow through a circuitous path created by the baffles to be filtered or treated.

In some embodiments, integrated tank 710 includes one or more monitoring units 714. Monitoring unit 714 may include one or more sensors of system 10. Monitoring unit 714 may send sensor signals to storage monitoring unit 650 for monitoring functions of the components or units of integrated tank 710. For example, a monitoring unit 714 may be configured to monitor a volume of a solution stored in reaction storage unit 110. A monitoring unit 714 or a sensor of monitoring unit 714 may be configured to monitor an amount of liquid received in waste storage unit 120 and/or a space remaining in waste storage unit 120. A monitoring unit 714 or a sensor of monitoring unit 714 may be configured to monitor usage time and/or an amount of filtering material in gas converter 130 and/or gas treatment unit 140.

System 10 as described herein may be used in various methods for generating and/or delivering NO gas. For example, system 10 may be used to generate NO gas on-demand for one or more treatment sessions. In some embodiments, system 10 may be used to provide a steady supply of NO gas at a predetermined concentration within a ramp period. A ramp period may refer to a transient period during which NO gas concentration of the product gas may change from an initial concentration to a predetermined concentration at the steady state. For example, during a ramp period, NO gas concentration of the product gas may increase from an initial concentration, such as zero, to a predetermined concentration at steady state, or may decrease form an initial concentration to a lower predetermined concentration at steady state. System 10 may be used to provide a steady NO gas flow over one or more treatment sessions or over one or more operating periods in a treatment session. System 10 may be used to reduce or minimize potential air pollution and/or exposure to toxic gases, such as NO₂, during the generation or delivery of NO gas.

In some embodiments, system 10 may perform an NO gas generation method. System 10 may convey a first solution (e.g., solution A) from reservoir 111 and a second solution (e.g., solution B) from reservoir 112 to a reaction chamber 208 of reaction module 200 (see FIG. 2A). System 10 may convey the first solution and second solution using two flow generators, respectively. System 10 may detect one or more conditions, such as the flow rate, of the first solution and the second solution using one or more sensors, such as two flow sensors, respectively. The first solution and the second solution may mix and react in the reaction chamber to generate NO gas.

System 10 may convey a carrier gas flow from carrier gas module 400 to a gas chamber 206 of reaction module 200 (see FIG. 2A). The carrier gas flow may transport NO gas diffused from reaction chamber 208 through a gas permeable membrane, such as an NO-permeable membrane, to gas chamber 206 and generate a product gas containing NO gas. Alternatively, system 10 may convey a carrier gas flow from carrier gas module 400 to a liquid region 2003 of a housing 2002 of reaction module 200 (see FIG. 2B). The carrier gas flow may sweep, purge, and/or entrain generated NO gas from the solution in liquid region 2003 to gas region 2004 and generate a product gas containing NO gas. System 10 may convey the generated product gas via an outlet circuit of reaction module 200 to treatment module 300. As described above, the outlet circuit may be disposed downstream of and in fluid communication with gas chamber 206 or gas region 2004 and disposed upstream of and in fluid communication with treatment module 300.

In one example, system 10 can generate NO gas by mixing a solution A that contains sodium nitrite and a solution B that contains citric acid. The two solutions can be conveyed to reaction unit 210 at a flow rate of about 0.2 m/min, and a carrier gas flow at a flow rate of about 1 L/min can convey the NO gas generated in reaction unit 210 in a product gas out of reaction unit 210. Solution A can have a sodium nitrite concentration of about 7.2 wt % and solution B can have a citric acid concentration of about 3.6 wt %, i.e., a mass concentration ratio between solution A and solution B of about 1:0.5. Varying the mass concentration ratio of Solutions A and B may change the NO gas concentration in the product gas, as shown in the Table 1 below. As shown below, increasing the concentration of citric acid in Solution B can increase NO gas concentration.

TABLE 1
NO gas concentration in product gas generated
by mixing sodium nitrite solution and citric
acid solution at different mass concentration ratios

| Mass | | | | |
|---|---|---|---|---|
| concentration ratio | | | | |
| of sodium nitrite | | | | |
| over citric acid | 1:0.25 | 1:0.5 | 1:1 | 1:2 |
| NO (ppm) | 412.0 | 608.7 | 815.6 | 950.7 |
| NOₓ (ppm) | 497.6 | 736.4 | 1049.2 | 1034.1 |
| NO₂ (ppm) | 85.6 | 127.7 | 233.5 | 83.3 |

In one example, system 10 can generate NO gas by mixing a solution A that contains sodium nitrite and a solution B that contains an acid. The two solutions can be conveyed to reaction unit 210 at a flow rate of about 0.2 mL/min, and a carrier gas flow at a flow rate of about 1 L/min can convey the NO gas generated in reaction unit 210 in a product gas out of reaction unit 210. Solution A can have a sodium nitrite concentration of about 7.2 wt % and solution B can have an acid concentration of 3.6 wt %. As shown in Table 2 below, NO gas concentration in the product gas generally increases as pH of solution B decreases.

TABLE 2
NO gas concentration in product gas generated by mixing sodium nitrite solution and
different acid solutions

| | Citric | Hydrochloric | Sulfuric | Acetic | Oxalic | L-Ascorbic | L-Maleic |
|---|---|---|---|---|---|---|---|
| Acid | Acid | Acid | Acid | Acid | Acid | Acid | Acid |
| NO | 594.1 | 1161.7 | 1053.4 | 451.6 | 1053.0 | 170.6 | 670.4 |
| (ppm) | | | | | | | |
| NOₓ | 743.2 | 1590.7 | 1480.2 | 611.3 | 1549.1 | 215.6 | 967.3 |
| (ppm) | | | | | | | |
| NO₂ | 149.1 | 429.0 | 426.7 | 159.7 | 496.1 | 44.9 | 296.9 |
| (ppm) | | | | | | | |
| pH | 1.9 | 0.22 | 0.51 | 2.24 | 0.92 | 2.25 | 1.91 |

In one example, system 10 can generate NO gas by mixing a solution A that contains sodium nitrite and a solution B that contains an acidic buffer. The two solutions can be conveyed to reaction unit 210 at a flow rate of 0.2 mL/min, and a carrier gas flow at a flow rate of 1 L/min can convey the NO gas generated in reaction unit 210 in a product gas out of reaction unit 210. Solution A can have a sodium nitrite concentration of 7.2 wt % and solution B can have a pH at or around 3. As shown in Table 3 below, NO gas concentration in the product gas generally increases as pH of the mixed solution decreases.

TABLE 3
NO gas concentration in the product gas generated by mixing sodium nitrite solution
and different acid buffer solutions.

| | | | | | Disodium | | |
|---|---|---|---|---|---|---|---|
| | | | | | Hydrogen | | |
| | | Sodium | Citric | | Phosphate- | Phthalic | |
| | | citrate- | Acid- | Glycine- | Sodium | acid- | HPES- |
| Acid | Citric | Hydrochloric | Sodium | Hydrochloric | Dihydrogen | Hydrochloric | Hydrochloric |
| buffer | Acid | acid | Citrate | acid | Phosphate | acid | acid |
| NO | 630.3 | 278.1 | 251.6 | 40.4 | 66.4 | 122.3 | 91.6 |
| (ppm) | | | | | | | |
| NOₓ | 775.8 | 331.1 | 297.7 | 61.6 | 86.2 | 149.8 | 112.1 |
| (ppm) | | | | | | | |
| NO₂ | 145.5 | 53.0 | 46.1 | 21.2 | 19.8 | 27.5 | 20.5 |
| (ppm) | | | | | | | |
| pH | 1.9 | 3.11 | 3.18 | 3.11 | 4.8 | 3.19 | 4.29 |

In some embodiments, the method for generating NO gas may include reducing or filtering one or more impurities in the product gas before the product gas conveyed to the treatment module. The one or more impurities may include at least one of moisture, solid matter, and other nitrogen oxides, such as NO₂ gas. The product gas may pass through gas converter 130, which may reduce or filter NO₂ gas, such as by converting NO₂ gas to NO gas. Gas converter 130 may increase NO gas concentration in the product gas.

In some embodiments, the method for generating NO gas may include detecting, by at least one gas sensor, a concentration of at least one of NO gas and NO₂ gas in the product gas before the product gas is conveyed to treatment module 300. For example, NO gas sensor 231 and NO₂ gas sensor 232 may be disposed upstream of treatment module 300. In some embodiments, the method for generating NO gas may include delivering the product gas a treatment site via a portable applicator of the treatment module. In other embodiments, the method for generating NO gas may include conveying the product gas to a treatment chamber configured to cover or immerse a treatment site. The method may further include conveying the product gas from the treatment chamber to gas treatment unit 140 to reduce or remove one or more nitrogen oxides, such as NO gas and NO₂ gas, before the product gas is released to the atmosphere. The method may further include recirculating the product gas with respect to the treatment chamber and adjusting the flow rate of the recirculated product gas to adjust the concentration of NO gas in the treatment chamber. As described herein, the terms “adjust” and “regulate” includes both increasing or decreasing a property or quantity towards a desired value.

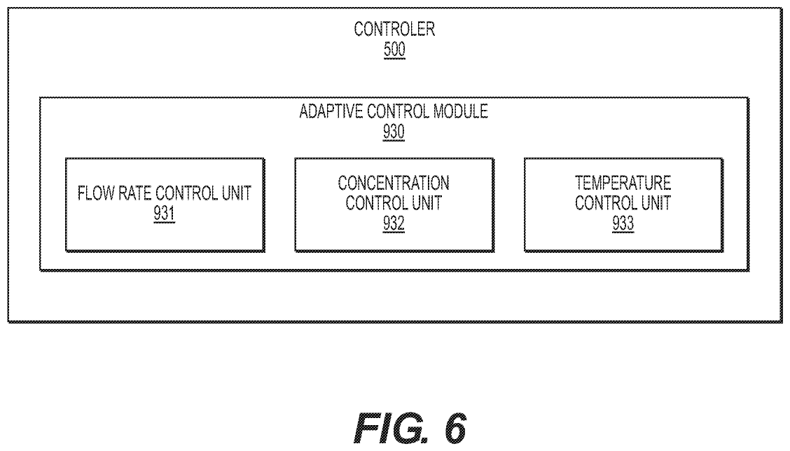

FIG. 6 is a schematic representation of an adaptive control module 930 of an NO gas generation system for controlling NO gas generation, according to some embodiments of the present disclosure. In some embodiments, system 10 includes controller 500 that includes computer-readable medium 510 and processor 520 (see FIG. 1). Controller 500 may be in communication with one or more units, modules, sensors, flow generators, or flow controllers of system to control and regulate NO gas generation (see FIGS. 1 and 6). Computer-readable medium 510 of controller 500 may store one or more sets of instructions that, when executed by the processor of controller 500, cause the processor to control system 10 to adjust one or more properties, such as concentration, flow rate, and duration of NO gas in the output product gas. Computer-readable medium 510 of controller 500 may store an adaptive control module 930 that includes instructions that, when executed by the processor of controller 500, cause the processor to perform control of an NO gas generation process 900.

Adaptive control module 930 may control an NO gas generation process 900 by adaptively controlling components of system 10 to adjust the concentration, duration, and/or flow rate of the generated NO gas in real time based on signals from one or more sensors of system 10, such as gas sensors, flow sensors, and temperature sensors. In some embodiments, adaptive control module 930 includes a flow rate control unit 931, a concentration control unit 932, and a temperature control unit 933. Each unit may include a set of instructions that can be executed by the processor of controller 500.

In some embodiments, flow rate control unit 931 may control one or more flow generators of system 10, such as flow generators 241, 242, and 430. In some embodiments, flow rate control unit 931 may receive one or more flow rate measurements from one or more flow sensors of system 10, such as flow sensor 233, 234, 450, and may control and adjust one or more flow generators of system 10 based on the received flow rate measurements. For example, flow rate control unit 931 may send instructions to flow generators 241 and 242 to adjust flow rates of solutions A and B based on flow rates measured by flow sensors 233 and 234, respectively. Also, flow rate control unit 931 may send instructions to flow generator 430 to adjust the flow rate of the carrier gas based on the flow measured by flow sensor 450.

In some embodiments, concentration control unit 932 may control one or more flow generators of system 10, such as flow generators 241, 242, 260, 430, and 351 of system 10. In some embodiments, concentration control unit 932 may receive one or more concentration measurements from one or more gas sensors of system 10, such as gas sensors 231 and 232, and may control and adjust one or more flow generators of system 10 based on the received concentration measurements. The concentration of NO gas in the product gas may be proportional to the flow rates of solutions A and B and may be inversely proportional to the flow rate of the carrier gas for conveying the generated NO gas out of reaction module 200. Based on detected concentration of NO gas measured by sensor 231, concentration control unit 932 may send instructions to flow generators 241 and 242 to adjust flow rates of solutions A and B and/or may send instructions to flow generator 430 to adjust the flow rate of the carrier gas flow entering reaction unit 210. Additionally, or alternatively, concentration control unit 932 may send instructions to flow generator 260 to adjust the flow rate of the recirculated liquid (e.g., a portion of mixed solutions A and B) relative to reaction unit 210. Adjusting the flow rate of the recirculated fluid may allow NO gas concentration in the product gas to reach a steady state in a shorter period.

In some embodiments, concentration control unit 932 may use a feedback control algorithm, such as a proportional-integral-derivative (PID) algorithm, to adjust the flow rates of one or more flow generators to adjust the concentration of NO gas in the product gas of reaction module 200, such as the product gas in the outlet circuit of or the product gas output by reaction module 200. As described herein, a feedback control algorithm, such as a PID algorithm, is a control algorithm that uses a current output of a system and a desired or expected output as the inputs to one or more control functions involving proportion, integration, and differentiation to adjust the real time output of the system to be at or close to the desired or expected value. In some embodiments, the differences between previous 20 times error values of the current measured output concentration can be used. A desired or expected output of system 10 may be a value or range of NO gas concentration at a certain flow rate predetermined as desirable for a particular treatment or treatment session. System 10 may use the feedback control algorithm to adjust the flow rates of flow generators, 241, 242, and 430, such as by adjusting the duty cycles of the flow generators, to adjust the output NO gas concentration of system 10 to a desired value or range. The present disclosure may use the PID algorithm as a feedback control algorithm. Other suitable feedback control algorithms may alternatively be used.

In some embodiments, a current measurement of NO gas concentration, an expected NO gas concentration, and the differences between a number of NO gas concentration measurements and the current measurement, such as about 10-20 measurements are used as input to the feedback control algorithm of system 10. The flow rates of one or more flow generators, such as flow generators 241, 242, 260, and 430, may be adjusted and controlled using the PID algorithm based on the input. Increasing the duty cycle of the PID algorithm may increase the flow rate of the flow generators, which may adjust, such as increase or decrease, the NO gas concentration to the predetermined valve.

In some embodiments, NO gas concentration of the product gas output from reaction module 200 may range from about 50 ppm to about 1500 ppm, such as from about 50 ppm to about 500 ppm, from about 50 ppm to about 1000 ppm, from about 100 ppm to about 500 ppm, from about 100 ppm to about 1000 ppm, from about 100 ppm to about 1500 ppm, from about 200 ppm to about 500 ppm, from about 200 ppm to about 1000 ppm, from about 200 ppm to about 1500 ppm, from about 400 ppm to about 1000 ppm, from about 400 ppm to about 800 ppm, from about 400 ppm to about 1200 ppm, from about 400 ppm to about 1500 ppm, from about 600 ppm to about 1000 ppm, from about 600 ppm to about 1200 ppm, from about 600 ppm to about 1500 ppm, from about 800 ppm to about 1000 ppm, from about 800 ppm to about 1200 ppm, from about 800 ppm to about 1500 ppm, from about 800 ppm to about 1000 ppm, from about 800 ppm to about 1200 ppm, and from about 1000 ppm to about 1500 ppm. The flow rate of the product gas output from reaction module 200 may range from about 1 L/min to about 10 L/min, such as from about 1 L/min to about 5 L/min, from about 5 L/min to about 8 L/min, and from about 5 L/min to about 10 L/min. In some embodiments, concentration control unit 932 may determine a difference between NO gas concentration of the product gas output from reaction module 200 and a predetermined NO gas concentration, and may adjust the flow rates of flow generators, 241, 242, and 430, to adjust the output NO gas concentration of the product gas output from reaction module 200 to be within an acceptable error range of the predetermined NO gas concentration, such as equal to or less than about 5%, 8%, or 10%.

In some embodiments, concentration control unit 932 may include a two-step process to adjust the concentration of NO gas in the product gas of reaction module 200. The two-step process may be an iterative process.

In some embodiments, in a first step of the two-step process, a flow sensor of sensing unit 230 may measure a flow rate of the product gas in the outlet circuit of reaction module 200. Concentration control unit 932 may determine whether the measured flow rate is within a predetermined flow rate range, which may correspond to a predetermined NO gas concentration for treatment. In response to determining that the measured flow rate is within the predetermined flow rate range, concentration control unit 932 may adjust flow generator 430 to adjust the concentration of NO gas in the product gas to the predetermined concentration using the PID algorithm. However, in response to determining that the measured flow rate is out of the predetermined flow rate range, concentration control unit 932 may proceed to the second step, where concentration control unit 932 may adjust flow generators 241, 242 to adjust the concentration of NO gas in the product gas using the PID algorithm until the measured flow rate comes within the predetermined flow rate range for the predetermined NO gas concentration. Concentration control unit 932 then may return to the first step to further adjust flow generator 430 to adjust the concentration of NO gas in the product gas until the difference between the NO gas concentration and the predetermined concentration is acceptable or minimized. In some embodiments, concentration control unit 932 may control and adjust the flow rate of flow generator 260. For example, when NO gas concentration in the product gas is higher than a predetermined concentration, and the flow rates of flow generators 241, 242 and/or 430 may not be further adjusted, concentration control unit 932 may reduce the flow rate of flow generator 260 to reduce NO gas concentration in the product gas. In some embodiments, concentration control unit 932 may turn off flow generator 260 to quickly reduce NO gas concentration in the product gas.

Concentration control unit 932 may repeat the two-step process to adjust the concentration of NO gas in the product gas and/or the flow rate of the product gas to predetermined values or ranges. The two-step process may allow reaction module 200 to output a product gas with a steady NO gas concentration within a predetermined range acceptable for treatment, and also may allow for reducing consumption of solutions for the same amount and concentration of NO gas generated, thereby increasing the service life of the solutions in reaction unit 210 before they are replaced or replenished.

In some embodiments, system 10 may include one or more temperature sensors configured to detect the temperature of one or more liquid flows in system 10, such as the flows of solutions A and/or B in reaction module 200. In some embodiments, sensors 233 and 234 may include temperature sensors configured to detect the temperature of the solutions conveyed from reactant storage unit 110. Temperature control unit 933 may receive one or more flow rate measurements from one or more temperature sensors of system 10, such as temperature sensors 113, 114, 233, and 234, and may control and adjust one or more flow generators of system 10 based on the received temperature measurements to adjust the concentration of NO gas in the product gas. Temperature control unit 933 also may use a PID algorithm to adjust the flow rates of one or more flow generators to adjust the concentration of NO gas in the product gas.

An adaptive control module 930 may allow control of various modules, units, and components of system 10 to increase stability and accuracy of the concentration and/or flow rate of NO gas output by system 10 over a treatment session.

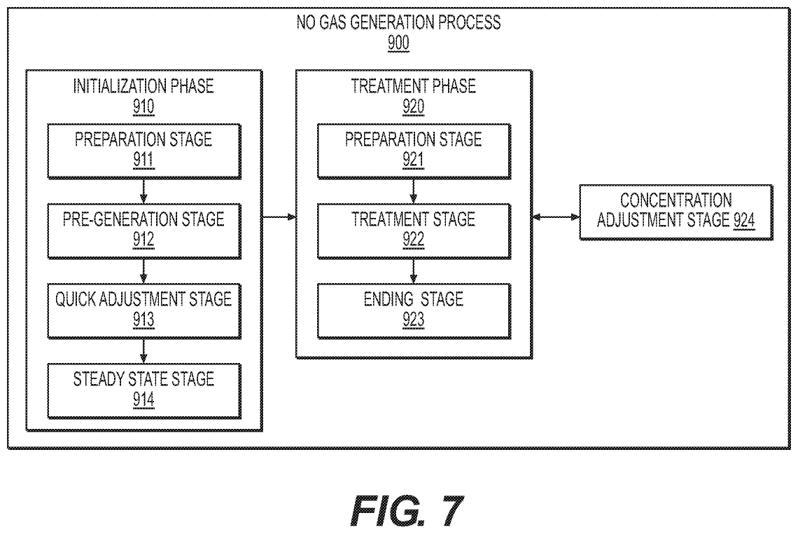

FIG. 7 is a schematic representation of an NO gas generation process 900, according to some embodiments of the present disclosure. NO gas generation process 900 is performed by one or more modules of system 10. One or more steps of NO gas generation process 900 are performed by controller 500 of system 10. In some embodiments, NO gas generation process 900 includes an initialization phase 910 and a treatment phase 920 following the initialization phase 910. Initialization phase 910 may initialize and prepare system 10 for NO generation and treatment phase 920 may generate NO gas for treatment as described below.

In some embodiments, initialization phase 910 may include one or more stages, such as a preparation stage 911, a pre-generation stage 912, a quick adjustment stage 913, and a steady state stage 914. These stages are described below with reference to FIGS. 1 and 2. As described herein, flow paths or circuits for transporting liquid flows in system 10 may be referred to as liquid circuits, such as the liquid recirculation circuit relative to reaction unit 210, and flow paths or circuits for transporting gas flows in system 10 may be referred to as gas circuits, such as the outlet circuit of reaction module 200.

In some instances, residual gas may exist or remain in system 10, such as NO gas or NO₂ gas from previous use of system 10. In some embodiments, after system 10 is powered on, NO gas generation process 900 starts preparation stage 911 to detect and remove, purge, or sweep gas remaining in system 10. Flow controllers 460, 470, 270, and/or 290 (see FIG. 2) may open to allow the remaining gas to flow through and exit system 10. For example, to sweep reaction module 200, flow controllers 460, 270, and 290 may open, and flow controllers 470, 280, and 340 may close. For example, to sweep treatment module 300, flow controllers 470, 280, and 340 may open, and flow controllers 460, 270, and 290 may close. Flow generator 430 of carrier gas module 400 may generate a carrier gas flow to sweep reaction module 200 or treatment module 300. A flow rate of the carrier gas may be equal to or less than about 10 L/min. At least some of the gas remaining or existing in system 10, such as in the gas circuits of reaction module 200 and/or treatment module 300, may be swept through flow controllers 270 and 290 to gas treatment unit 140 of storage module 100 and discharged from system 10 to the atmosphere. Gas treatment unit 140 may absorb and/or reduce toxic nitrogen oxides in the remaining gas before discharging the remaining gas to the atmosphere. Flow controller 360 disposed downstream of flow controllers 280 and 290 may be a one-way valve configured to direct the remaining gas flow to filter through gas treatment unit 140.

In some embodiments, sensing unit 230 may measure concentrations of NO gas and/or NO₂ gas in the remaining gas in the gas circuits of system 10 at preparation stage 911. Controller 500 may determine whether the remaining gas in system 10 has been reduced to an acceptable level or exhausted from system 10 based on the measured gas concentrations. Controller 500 may determine that preparation stage 911 is complete when the concentration of NO gas in system 10 has been reduced to be at or below a preset threshold. The preset threshold may be lower than the predetermined NO gas concentration. Additionally, and alternatively, controller 500 may determine that preparation stage 911 is complete after a predetermined period of time. The predetermined period of time may be equal to or less than about 1 minute. For example, it may be about 5 seconds, about 10 seconds, about 20 seconds, about 30 seconds, about 45 seconds, or about 50 seconds. In some embodiments, controller 500 may determine that preparation stage 911 is complete after determining that NO gas concentration reached a predetermined concentration or within an acceptable range of the predetermined concentration. When preparation stage 911 is complete, NO gas generation process 900 may proceed to pre-generation stage 912.

In some embodiments, at preparation stage 911, controller 500 may determine various control parameters of pre-generation stage 912. Pre-generation stage 912 may sweep remaining gas out of system 10 and may prefill the liquid circuits of system 10 with solutions to quickly increase an initial NO gas concentration to a predetermined steady state concentration.

In some embodiments, pre-generation stage 912 includes two operations. In a first operation, as in preparation stage 911, system 10 is configured to detect and remove, purge, or sweep remaining gas out of system 10. In a second operation, flow control unit 240 may transport one or more solutions to reaction unit 210 to start NO generation. For example, in the second operation, flow generators 241 and 242 may transport solutions A and B from storage module 100 to reaction unit 210. In some embodiments, flow generators 241 and 242 are set at the same flow rate. Separation device 220 may receive a mixed solution and NO gas from reaction unit 210, at least a portion of which may be conveyed to waste storage unit 120. Thus, at pre-generation stage 912, one or more of the liquid circuits of system 10 can be primed with liquid to prepare for or facilitate quick ramping up of NO gas concentration in the product gas in the next stage.

Pre-generation stage 912 may proceed from the first operation to the second operation after a duration of the first operation that may be equal to or less than about 1 minute. For example, it may be about 5 seconds, about 10 seconds, about 20 seconds, about 30 seconds, about 45 seconds, or about 50 seconds. A duration of the second operation may be equal to or less than about 1 minute. For example, it may be about 5 seconds, about 10 seconds, about 20 seconds, about 30 seconds, about 45 seconds, or about 50 seconds. In some embodiments, the second operation may be completed when a measured NO gas concentration in the outlet circuit of reaction module 200 reaches a predetermined NO gas concentration or concentration range. The flow rates of flow generators 241 and 242 during the second operation may range from about 0 mL/min to about 10 mL/min.

In some embodiments, NO gas generation process 900 may proceed from pre-generation stage 912 to quick adjustment stage 913 to increase NO gas concentration in a short ramp period. At quick adjustment stage 913, as in pre-generation stage 912, flow generators, such as flow generators 241, 242, and 430, of system 10 may continue to operate to transport one or more solutions from storage module 100 and a carrier gas flow from carrier gas module 400 to reaction unit 210 to continue NO generation. At quick adjustment stage 913, separation device 220 may receive a mixed solution and NO gas from reaction unit 210. A flow generator 260 may recirculate a portion of the received mixed solution separated by separation device 220 relative to reaction unit 210 by flow generator 260 (see FIG. 2). Another portion of the received mixed solution and the received NO gas separated by separation device 220 may be conveyed to waste storage unit 120. The recirculation of a portion of the mixed solution may facilitate mixing of the solutions in reaction unit 210 and allow for quick ramping up of NO gas concentration in the product gas of reaction module 200.

The duration of quick adjustment stage 913 and/or flow rate of flow generator 260 at quick adjustment stage 913 may depend on the predetermined NO concentration or concentration range. For example, a predetermined NO gas concentration range may be from about 100 ppm to about 900 ppm. In some embodiments, a duration of quick adjustment stage 913 may be equal to or less than about 3 minutes. For example, it may be about 30 seconds, about 45 seconds, about 60 seconds, about 90 seconds, about 2 minutes, or about 2.5 minutes. In some embodiments, the flow rate of flow generator 260 may range from about 0 L/min to about 3 L/min. For example, it may be about 0.5 L/min, about 1.0 L/min, about 1.5 L/min, about 2.0 L/min, or about 2.5 L/min.

In some embodiments, NO generation process 900 may proceed from quick adjustment stage 913 to steady state stage 914. At steady state stage 914, NO gas concentration in the gas circuits of system 10, such as the outlet circuit of reaction module 200, may quickly stabilize at a predetermined concentration or within a predetermined concentration range. At steady state stage 914, as in quick adjustment stage 913, flow generators, such as flow generators 241, 242, and 430, of system 10 may continue to operate to transport one or more solutions from storage module 100 and a carrier gas flow from carrier gas module 400 to reaction unit 210 to continue NO generation.

In some embodiments, steady state stage 914 may include two operations. In a first operation, based on a predetermined NO gas concentration or concentration range, flow rates of flow generators 241 and 242 may be adjusted while the flow rate of flow generator 260 may remain unchanged for a pre-determined period, such as equal to or less than about 5 minutes. For example, it may be about 30 seconds, about 60 seconds, about 90 seconds, about 2 minutes, about 2.5 minutes, about 3 minutes, about 3.5 minutes, about 4 minutes, or about 4.5 minutes. Flow generators 241 and 242 may be adjusted to the same flow rate. After the pre-determined period, in a second operation, NO gas sensor 231 may measure NO gas concentration. A PID algorithm may be used to adjust flow rate of flow generator 430 based on the difference between the measured NO gas concentration and a predetermined NO gas concentration for treatment over an adjustment time period, during which the flow rates of flow generators 241, 242, and 260 may not be changed.

In some embodiments, NO generation process 900 may proceed to treatment phase 920 when one or more criteria are met during steady state stage 914. For example, NO generation process 900 may proceed to treatment phase 920 when the period of steady state stage 914 passes a predetermined duration, such as based on a counter in controller 500. For example, for a predetermined NO gas concentration range from about 100 ppm to about 900 ppm, the flow rates of flow generators 241 and 242 may range from about 0 L/min to about 0.3 mL/min, the flow rate of flow generator 430 may range from about 0 L/min to about 3 L/min, and the duration of steady state stage 914 may be equal to or less than about 5 minutes. For example, the flow rates of flow generators 241 and 242 may be about 0.05 mL/min, about 0.1 mL/min, about 0.15 mL/min, about 0.2 mL/min, or about 0.25 mL/min. The flow rate of flow generator 430 may be about 0.5 L/min, about 1.0 L/min, about 1.5 L/min, about 2.0 L/min, or about 2.5 L/min. The duration of steady state stage 914 may be about 30 seconds, about 60 seconds, about 90 seconds, about 2 minutes, about 2.5 minutes, about 3 minutes, about 3.5 minutes, about 4 minutes, or about 4.5 minutes. Additionally, or alternatively, NO generation process 900 may proceed to treatment phase 920 when the steady state NO gas concentration measured by the NO gas sensor deviates from the predetermined NO gas concentration or concentration range within an acceptable error range, such as from about 0% to about 10% deviation.

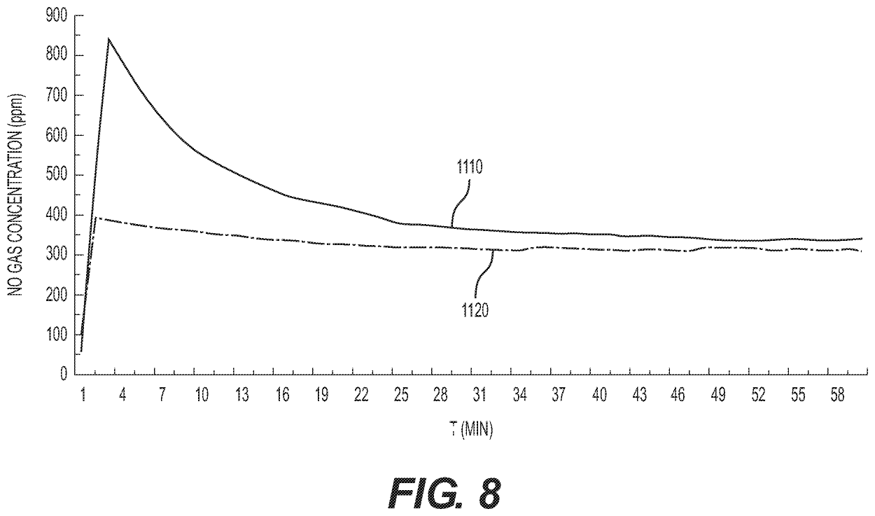

FIG. 8 is a graphical representation of NO gas concentration over time during an NO gas generation process, according to some embodiments of the present disclosure. The NO gas concentration may be measured by gas sensor 231 disposed at the outlet circuit of reaction module 200. Line 1110 illustrates a change of NO gas concentration measured over time without implementing initialization phase 910, and line 1120 illustrates a change of NO gas concentration measured over time while implementing initialization phase 910. NO gas concentration reached a steady state concentration due to the adaptive control during initialization phase 910 within about 5 minutes, which reduced the amounts of solutions consumed for initializing system 10 for further generating NO gas for treatment, and improved efficiency and stability of the NO gas concentration for providing more accurate NO gas treatment.

In some embodiments, NO generation process 900 may proceed from initialization phase 910 to treatment phase 920. In some embodiments, treatment phase 920 may include one or more stages, such as a preparation stage 921, a treatment stage 922, and a termination stage 923. These stages are described below with reference to FIGS. 1-4B. Treatment phase 920 may include one or more stages based on the type of treatment device 330 in treatment module 300, such as portable applicator 370 or treatment chamber 380. In some embodiments, preparation stage 921 may be performed before, during, or after preparation stage 911 in initialization phase 910. In such instances, system 10 may complete preparation stage 921 at the end of initialization phase 910, and system 10 may perform treatment stage 922 after initialization phase 910 is implemented.

In some embodiments, treatment module 300 includes portable applicator 370 and treatment phase 920 may include treatment stage 922 and termination stage 923. At treatment stage 922, as in steady state stage 914, flow generators, such as flow generators 241, 242, and 430, of system 10 may continue to operate to transport one or more solutions from storage module 100 and a carrier gas flow from carrier gas module 400 to reaction unit 210 to continue NO generation. At treatment stage 922, one or more flow controllers are configured to allow the product gas containing NO gas to flow into portable applicator 370 for delivering NO gas to a treatment site. For example, flow controllers 470 and 290 may be closed (see FIGS. 2A and 2B). Flow controllers 270 and 280 may be opened to direct the product gas flow from gas outlet 212, through the outlet circuit, to treatment device 330, i.e., portable applicator 370 in this example. At treatment stage 922, adaptive control module 930 may control the concentration and/or flow rate of the generated NO gas as described above. Depending on the particular treatment or treatment session, treatment stage 922 may last from about 1 minute to about 1 hour, for example. For example, treatment stage 922 may last for about 5 minutes, about 10 minutes, about 12 minutes, about 20 minutes, about 30 minutes, about 45 minutes, or about 60 minutes.

Termination stage 923 may follow treatment stage 922. At termination stage 923, flow generators, such as flow generators 241, 242, and 260 of system 10 may stop to operate or reduce to zero flow rate. Flow controllers may be configured to terminate conveying the product gas to treatment module 300. Flow controller 280 may be closed. Flow generator 430 may continue to operate and flow controller 460 may remain open to sweep or purge remaining gas in reaction module 200, such as in reaction unit 210, separation device 220, and/or the outlet circuit. Flow controller 290 may be opened to allow the remaining gas swept or purged from reaction module 200 to be conveyed to gas treatment unit 140 before being discharged from system 10, such as to the atmosphere. After purging or sweeping the remaining gas for a period of time, one or more flow controllers of system 10 may be closed. For example, flow controllers 460, 470, 280, and/or 290 may be closed and system 10 may be turned off or prepare for a next NO generation process.

In some embodiments, treatment module 300 includes treatment chamber 380, and treatment phase 920 may include preparation stage 921, treatment stage 922, and termination stage 923. Preparation stage 921 may allow NO gas to distribute in treatment chamber 380, purge preexisting or residue gas in treatment chamber 380, and/or may allow NO gas concentration in treatment chamber 380 to reach a steady state concentration at or around a predetermined NO gas concentration or concentration range for treatment.

In some embodiments, at preparation stage 921, as in steady state stage 914, flow generators, such as flow generators 241, 242, and 430, of system 10 may continue to operate to transport one or more solutions from storage module 100 and a carrier gas flow from carrier gas module 400 to reaction unit 210 to continue NO generation. Additionally, one or more flow controllers are configured to allow the product gas containing NO gas to flow into treatment chamber 380 for delivering NO gas to a treatment site and to recover the NO gas from treatment chamber 380. For example, flow controllers 470 and 290 may be closed (see FIG. 2). Flow controllers 270 and 280 may be opened to direct the product gas flow from gas outlet 212, through the outlet circuit, to treatment device 330, i.e., treatment chamber 380 in this example. Flow controller 320 may be opened to allow the product gas accumulated in treatment chamber 380 to flow back via recovery circuit 340 to gas treatment unit 140.

In some embodiments, at preparation stage 921, flow controller 352 is opened and flow generator 351 generates a recirculated product gas flow relative to treatment chamber 380. The flow rate of flow generator 351 may range from about 0 L/min to about 5 L/min. In some embodiments, at preparation stage 921, NO gas concentration in treatment chamber 380 may reach a steady state NO gas concentration and treatment phase 920 may proceed from preparation stage 921 to treatment stage 922. The steady state NO gas concentration in treatment chamber 380 may be at or close to a predetermined NO gas concentration for treatment within an acceptable range.

In some embodiments, at treatment stage 922, as described above, one or more flow controllers are configured to continue NO generation, conveying NO gas to treatment chamber 380 for surrounding or immersing a treatment site. Flow controllers 270 and 280 may be opened to direct the product gas flow from gas outlet 212, through the outlet circuit, to treatment chamber 380 (see FIG. 2). At treatment stage 922, flow generator 351 may continue to operate to generate a recirculated product gas flow relative to treatment chamber 380. Flow generator 351 may operate for a period of time equal to or less than about 1 hour. For example, flow generator 351 may operate for about for about 5 minutes, about 10 minutes, about 12 minutes, about 20 minutes, about 30 minutes, about 45 minutes, or about 60 minutes. At treatment stage 922, adaptive control module 930 may control the flow rate of the flow generators, such as flow generators 241, 242, 260, 430, and 351, and adjust the concentration and/or flow rate of the generated NO gas as described above.

In some embodiments, at termination stage 923, as described above, flow generators, such as flow generators 241, 242, and 260 of system 10, may stop to operate or reduce to zero flow rate. Flow generator 430 may continue to operate and flow controllers 460, 270, and 290 may open to sweep or purge remaining gas in reaction module 200, such as in reaction unit 210, separation device 220, and the outlet circuit of reaction module 200. Alternatively, or additionally, flow controllers 460, 270, and 290 may be closed. Flow controllers 470 and 280 may open to allow sweeping or purging remaining gas from treatment chamber 380 to gas treatment unit 140 before being discharged from system 10, such as to the atmosphere. Flow controller 320 may open such that the purged product gas would be conveyed to gas treatment unit 140 before being discharged from system 10. After purging or sweeping the remaining gas for a period of time, flow generators of system 10 may stop to operate and flow controllers of system 10 may be closed at the same time or in series. For example, flow generators, such as flow generators 241, 242, and 260 of system 10, may stop to operate or reduce to zero flow rate, and flow controllers 470, 280, and 351 may be closed. System 10 may then be turned off or prepare for a next NO generation process.

In some embodiments, treatment phase 920 includes one or more concentration adjustment stages 924 to quickly adjust the NO gas concentration in the product gas. Concentration adjustment stage 924 may be implemented due to a deviation of the NO gas concentration from a predetermined NO gas concentration for treatment, a change of predetermined NO gas concentration as required by a treatment protocol, or a change of predetermined NO gas concentration as instructed by a user of system 10. In some embodiments, concentration adjustment stage 924 includes a ramp period and a steady state period. The ramp period may be a ramp-up period or a ramp-down period.

In some embodiments, in response to determining NO gas concentration in the product gas output by reaction module 200 or treatment module 300 is lower than a predetermined NO gas concentration, controller 500 may implement a ramp-up period of concentration adjustment stage 924. During the ramp-up period, flow rates of flow generators 241, 242 may be increased and/or flow rate of flow generator 430 may be decreased to increase NO gas concentration in the product gas. For example, based on the NO gas concentration measured by NO gas sensor 231, adaptive control module 930 may adjust the flow rates of flow generators 241, 242 in the range from about 0 mL/min to about 0.3 mL/min, and may adjust the flow rate of flow generator 430 in the range from about 0.1 L/min to about 4 L/min. For example, the flow rates of flow generators 241 and 242 may be about 0.05 mL/min, about 0.1 mL/min, about 0.15 mL/min, about 0.2 mL/min, or about 0.25 mL/min. The flow rate of flow generator 430 may be about 0.5 L/min, about 0.8 L/min, about 1.0 L/min, about 1.5 L/min, about 2.0 L/min, about 2.5 L/min, about 3.0 L/min, or about 3.5 L/min.

In some embodiments, in response to determining NO gas concentration in the product gas output by reaction module 200 or treatment module 300 is higher than a predetermined NO gas concentration, controller 500 may implement a ramp-down period of concentration adjustment stage 924. During the ramp-down period, flow rates of flow generators 241, 242 may be decreased or flow generators 241, 242, and/or 260 may be stopped for a brief pause period, depending on the amount of adjustment needed. The pause period may be equal to or less than about 10 minutes. For example, it may be about 1 minute, about 2 minutes, about 3 minutes, about 4 minutes, about 5 minutes, about 6 minutes, about 7 minutes, about 8 minutes, or about 9 minutes. After the pause period, the flow rates of flow generators 241, 242 may be increased or decreased based on a difference between measured NO gas concentration and the predetermined NO gas concentration. The flow rate of flow generator 430 also may be increased or decreased to adjust the NO gas concentration in the product gas towards the predetermined concentration. For example, based on the NO gas concentration measured by NO gas sensor 231, adaptive control module 930 may adjust the flow rates of flow generators 241, 242 after the pause period.

Concentration adjustment stage 924 may proceed from the ramp period to the steady state period after one or more criteria are met. For example, the steady state period may start when the NO gas concentration measured by the NO gas sensor is equal to or within a range around the predetermined NO gas concentration, such as from about 0% to about 20% deviation from the predetermined NO gas concentration. During the steady state period, based on the NO gas concentration measured by NO gas sensor 231, adaptive control module 930 may adjust flow generators of system 10 to adjust NO gas concentration in the product gas to be with in an acceptable range of deviation from the predetermined NO gas concentration. In some embodiments, the acceptable range of deviation may be from about 0% to about 10% deviation. For example, based on the NO gas concentration measured by NO gas sensor 231, adaptive control module 930 may adjust the flow rates of flow generators 241, 242, and/or flow generator 430 over a period of time.

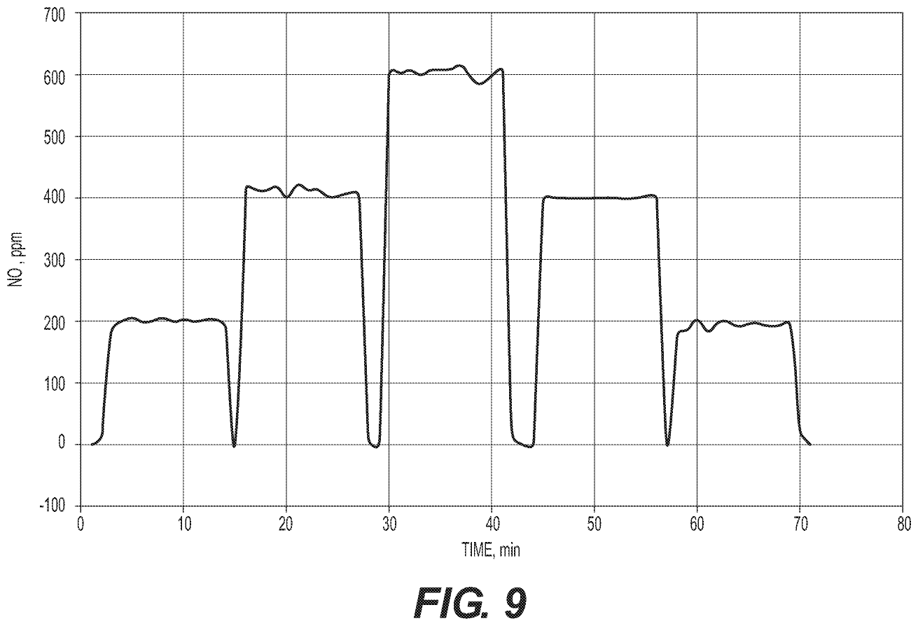

FIG. 9 is a graphical representation of NO gas concentration over time during an NO gas generation process 900, according to some embodiments of the present disclosure. NO gas generation process 900 may include one or more treatment periods with different predetermined NO gas concentrations for treatment. For example, FIG. 9 shows actual NO gas concentration over five treatment periods with predetermined NO gas concentration at 200 ppm, 400 ppm, 600 ppm, 400 ppm, and 200 ppm, respectively. Each treatment period may include one or more stages of initialization phase 910 and treatment phase 920. Treatment phase 920 may include one or more concentration adjustment stages 924, such as a ramp-up period or a ramp-down period. A ramp-up or ramp-down period allows NO gas concentration to quickly increase or decrease towards an expected value, e.g., a predetermined NO gas concentration. Concentration adjustment stage 924 may reduce consumption of solutions for adjusting NO gas concentration in the product gas and increase the service life of the solutions in reaction unit 210 before they are replaced or replenished. During each treatment period, after the NO gas concentration is increased or decreased, the NO gas concentration is quickly stabilized to the predetermined concentration within an acceptable error range, such as from about 0% to about 7% of the predetermined concentration.

As described herein, the steps of the disclosed methods may be modified in any manner, including by reordering steps, inserting, and/or deleting steps. One or more steps of the disclosed methods may be performed at the same time or in any suitable time sequence unless described otherwise.

The foregoing descriptions have been presented for purposes of illustration. They are not exhaustive and are not limited to precise forms or embodiments disclosed. Modifications and adaptations of the embodiments will be apparent from consideration of the specification and practice of the disclosed embodiments. In addition, while certain components have been described as being connected or in communication to one another, such components may be integrated with one another or distributed in any suitable fashion. A component described with a particular embodiment can be interchanged with another component in another embodiment.

Moreover, while illustrative embodiments have been described herein, the scope includes any and all embodiments having equivalent elements, modifications, omissions, combinations (e.g., of aspects across various embodiments), adaptations or alterations based on the present disclosure. Further, the steps of the disclosed methods or processes can be modified in any manner, including reordering steps or inserting or deleting steps.

It also can be appreciated that the above-described embodiments can be implemented by hardware, or software (program codes), or a combination of hardware and software. If implemented by software, it may be stored in the above-described computer-readable medium. The software, when executed by one or more processors, can perform at least some of the steps of the disclosed methods or processes.

1. A nitric oxide (NO) gas generation system, the system comprising:
a first reservoir configured to contain a first solution, the first solution comprising a nitrite source;
a second reservoir configured to contain a second solution, the second solution comprising an acidic solution;
a flow generator in fluid communication with the first reservoir and the second reservoir;
a housing configured to contain a reaction chamber and a gas chamber, the reaction chamber configured to contain a liquid and the gas chamber configured to receive gas from the reaction chamber;
the reaction chamber disposed downstream of and in fluid communication with the flow generator; and
a carrier gas source disposed upstream of and in fluid communication with the gas chamber.

2. The system of claim 1, further comprising:
a liquid separation tank disposed downstream of and in fluid communication with the reaction chamber, the liquid separation tank having a first outlet and a second outlet;
a first circulation circuit disposed downstream of and in fluid communication with the first outlet of the liquid separation tank and disposed upstream of and in fluid communication with the reaction chamber; or
a waste storage unit disposed downstream of and in fluid communication with the second outlet of the liquid separation tank.

3. The system of claim 1, further comprising an inlet circuit disposed upstream of and in fluid communication with the gas chamber of the housing and disposed downstream of and in fluid communication with the carrier gas source, the inlet circuit comprising at least one of a flow controller, a filter, a flow generator, a flow sensor, and a gas capacitor.

4. The system of claim 1, further comprising an outlet circuit disposed downstream of and in fluid communication with the gas chamber of the housing and disposed upstream of and in fluid communication with a treatment module, the outlet circuit configured to convey a product gas to the treatment module.

5. The system of claim 4, wherein the outlet circuit comprises at least one filter configured to reduce one or more impurities the product gas, the one or more impurities comprising at least one of moisture, solid matter, and nitrogen dioxide.

6. The system of claim 4, wherein the outlet circuit comprises at least one gas sensor configured to detect a concentration of at least one of NO gas and nitrogen dioxide (NO₂) gas in the product gas.

7. The system of claim 4, wherein the outlet circuit comprises at least one of a moisture sensor and a temperature sensor configured to detect a humidity and/or a temperature of the product gas.

8. The system of claim 4, wherein the treatment module comprises a portable applicator having an open end and configured to deliver the product gas to a treatment site.

9. The system of claim 8, wherein the portable applicator is a hand-held applicator and/or an annular applicator.

10. The system of claim 4, wherein the treatment module comprises a treatment chamber configured to contain the product gas and cover at least a portion of a treatment site.

11. The system of claim 10, wherein the treatment module comprises a gas treatment unit disposed downstream of the treatment chamber and configured to absorb at least one of NO gas and NO₂ gas.

12. The system of claim 10, wherein the treatment module further comprises a second circulation circuit configured to generate a fluid flow relative to the treatment chamber, the second circulation circuit comprising at least one of a flow generator, a flow controller, and a gas filter.

13. (canceled)

14. The system of claim 1, further comprising at least one sparger disposed in the liquid of the reaction chamber and in fluid communication with the carrier gas source, wherein the at least one sparger is configured to emanate bubbles of carrier gas from the carrier gas source.

15-16. (canceled)

17. A nitric oxide (NO) gas generation system, the system comprising:
a first reservoir configured to contain a first solution, the first solution comprising a nitrite source;
a second reservoir configured to contain a second solution, the second solution comprising an acidic solution;
a first flow generator in fluid communication with the first reservoir;
a second flow generator in fluid communication with the second reservoir;
a housing configured to contain a reaction chamber and a gas chamber, the gas chamber separated from the reaction chamber by a gas-permeable membrane, the reaction chamber disposed downstream of and in fluid communication with the first flow generator and the second flow generator;
a carrier gas source disposed upstream of and in fluid communication with the gas chamber;
a liquid separation tank disposed downstream of and in fluid communication with the reaction chamber, the liquid separation tank having a first outlet and a second outlet;
a first circulation circuit disposed downstream of and in fluid communication with the first outlet of the liquid separation tank and disposed upstream of and in fluid communication with the reaction chamber;
a waste storage unit disposed downstream of and in fluid communication with the second outlet of the liquid separation tank;
a treatment module disposed downstream of and in fluid communication with the gas chamber of the housing, the treatment module comprising
a treatment chamber configured to contain the product gas and cover at least a portion of a treatment site;
a gas treatment unit disposed downstream of the treatment chamber and configured to absorb NO gas; and
a second circulation circuit configured to generate a fluid flow relative to the treatment chamber.

18-31. (canceled)

32. The system of claim 1, wherein the gas chamber separated from the reaction chamber by a gas-permeable membrane.

33. The system of claim 1, wherein the flow generator comprises a first flow generator in fluid communication with the first reservoir, and a second flow generator in fluid communication with the second reservoir.
